(19) United States
(12) Patent Application Publication
(10) Pub. No.: US 2025/0049569 A1
(43) Pub. Date: Feb. 13, 2025

(54) TRANSCATHETER VALVE DELIVERY SYSTEM WITH IMPROVED LOADING AND DEPLOYMENT

(71) Applicant: MEDTRONIC, INC., Minneapolis, MN (US)

(72) Inventors: Kevin MAUCH, Windsor, CA (US); Alejandra CACERES, Healdsburg, CA (US); Nrupen ZAVER, Santa Rosa, CA (US); Mikayla NOVAK, Santa Rosa, CA (US)

(21) Appl. No.: 18/723,624

(22) PCT Filed: Jan. 20, 2023
(86) PCT No.: PCT/IB2023/050497

§ 371 Date: Jun. 24, 2024

Related U.S. Application Data
(60) Provisional application No. 63/304,022, filed on Jan. 28, 2022.

(51) Int. Cl. A61F 2/24

(52) U.S. Cl. CPC A61F 2/2436; A61F 2/2418

(57) ABSTRACT

A system includes a prosthesis and a delivery system for delivery thereof. The prosthesis includes a self-expanding frame and a plurality of attachment tabs extending from a first end of the frame. The delivery system includes a shaft, a piston disposed over the shaft, and a capsule which is movable relative to the piston. The piston includes an annular groove or a plurality of circumferentially-extending grooves on an outer surface thereof. In a delivery configuration of the delivery system, the plurality of attachment tabs are disposed within the annular groove of the piston, or the plurality of circumferentially-extending grooves of the piston, and the capsule covers and constrains the prosthesis in a radially collapsed configuration, with the capsule extending over the annular groove, or the plurality of circumferentially-extending grooves, of the piston and the attachment tabs received therein.

### FIELD

The present technology is generally related to systems and methods for transcatheter delivery and deployment of an implant or prosthesis to an annulus, such as a heart valve.

### BACKGROUND

A human heart includes four heart valves that determine the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped and blood flow is controlled by a heart-lung bypass machine.

More recently, minimally invasive approaches have been developed to facilitate catheter-based implantation of a prosthetic heart valve or prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

The heart valve prosthesis employed with catheter-based, or transcatheter, procedures generally includes an expandable multi-level frame or stent that supports a valve structure having a plurality of leaflets. The frame can be contracted during percutaneous transluminal delivery, and expanded upon deployment at or within the native valve. One type of valve stent can be initially provided in an expanded or un-crimped condition, then crimped or compressed about a balloon portion of a catheter. The balloon is subsequently inflated to expand and deploy the prosthetic heart valve. With other stented prosthetic heart valve designs, the stent frame is formed to be self-expanding. With these systems, the valved stent is crimped down to a desired size and held in that compressed state within a sheath for transluminal delivery. Retracting the sheath from this valved stent allows the stent to self-expand to a larger diameter, fixating at the native valve site. In more general terms, then, once the prosthetic valve is positioned at the treatment site, for instance within an incompetent native valve, the stent frame structure may be expanded to hold the prosthetic valve firmly in place.

The present disclosure addresses problems and limitations associated with the related art.

### SUMMARY

According to a first embodiment hereof, the disclosure provides a system including a valve prosthesis and a delivery system for percutaneously delivering the valve prosthesis. The valve prosthesis includes a frame and a prosthetic valve disposed within the frame, the frame being self-expanding and including a plurality of attachment tabs extending from a first end of the frame delivery system. The delivery system includes a shaft, a piston disposed over the shaft, and a capsule, the capsule being movable relative to the piston. The piston includes an annular groove on an outer surface thereof. In a delivery configuration of the delivery system, the plurality of attachment tabs are disposed within the annular groove of the piston and the capsule covers and constrains the valve prosthesis in a radially collapsed configuration, with the capsule extending over the annular groove of the piston and the attachment tabs received therein.

In an aspect of the first embodiment, and in combination with any other aspects herein, the disclosure provides that each attachment tab of the plurality of attachment tabs has an L-shaped configuration and includes a leg, a base, and a bend extending between the leg and the base. The base extends generally perpendicular to a longitudinal axis of the delivery system and is configured to extend into the annular groove of the piston.

In an aspect of the first embodiment, and in combination with any other aspects herein, the disclosure provides that the leg of each attachment tab of the plurality of attachment tabs extends generally parallel to the longitudinal axis of the delivery system.

In an aspect of the first embodiment, and in combination with any other aspects herein, the disclosure provides that the leg of each attachment tab of the plurality of attachment tabs extends at an acute angle relative to the longitudinal axis of the delivery system.

In an aspect of the first embodiment, and in combination with any other aspects herein, the disclosure provides that the bend of each attachment tab of the plurality of attachment tabs has a curved profile.

In an aspect of the first embodiment, and in combination with any other aspects herein, the disclosure provides that the bend of each attachment tab of the plurality of attachment tabs forms an angle between the leg and the base, the angle being between 80 and 100 degrees.

In an aspect of the first embodiment, and in combination with any other aspects herein, the disclosure provides that each attachment tab of the plurality of attachment tabs includes a spherical ball disposed at a free end thereof, the spherical ball being attached to the base.

According to a second embodiment hereof, the disclosure provides a system including a valve prosthesis and a delivery system for percutaneously delivering the valve prosthesis. The valve prosthesis includes a frame and a prosthetic valve disposed within the frame, the frame being self-expanding and including a plurality of attachment tabs extending from a first end of the frame. Each attachment tab of the plurality of attachment tabs has an L-shaped configuration and includes a leg, a base, and a bend extending between the leg and the base. The base extends generally perpendicular to a longitudinal axis of the delivery system and the base has a first length. The delivery system includes a shaft, a piston disposed over the shaft, and a capsule, the capsule being movable relative to the piston. The piston includes a plurality of circumferentially-extending grooves on an outer surface thereof. Each circumferentially-extending groove has a length that is at least 300% greater than the first length of the base of an attachment tab of the plurality of attachment tabs. In a delivery configuration of the delivery system, an attachment tab of the plurality of attachment tabs is disposed within a circumferentially-extending groove of the plurality of circumferentially-extending grooves and the capsule covers and constrains the valve prosthesis in a radially collapsed configuration, with the capsule extending over the plurality of circumferentially-extending grooves and the attachment tabs received therein.

In an aspect of the second embodiment, and in combination with any other aspects herein, the disclosure provides that the leg of each attachment tab of the plurality of attachment tabs extends generally parallel to the longitudinal axis of the delivery system.

In an aspect of the second embodiment, and in combination with any other aspects herein, the disclosure provides that the leg of each attachment tab of the plurality of attachment tabs extends at an acute angle relative to the longitudinal axis of the delivery system.

In an aspect of the second embodiment, and in combination with any other aspects herein, the disclosure provides that the bend of each attachment tab of the plurality of attachment tabs has a curved profile.

In an aspect of the second embodiment, and in combination with any other aspects herein, the disclosure provides that bend of each attachment tab of the plurality of attachment tabs forms an angle between the leg and the base, the angle being between 80 and 100 degrees.

In an aspect of the second embodiment, and in combination with any other aspects herein, the disclosure provides that each attachment tab of the plurality of attachment tabs includes a spherical ball disposed at a free end thereof, the spherical ball being attached to the base.

In an aspect of the second embodiment, and in combination with any other aspects herein, the disclosure provides that a number of the attachment tabs is equal to a number of circumferentially-extending grooves.

According to a third embodiment hereof, the disclosure provides a method of coupling a valve prosthesis to a delivery system for delivery thereof. A plurality of attachment tabs of the valve prosthesis is positioned into an annular groove of a piston of the delivery system. The valve prosthesis includes a frame and a prosthetic valve disposed within the frame, the frame being self-expanding and including the plurality of attachment tabs extending from a first end of the frame. At least a portion of the valve prosthesis is positioned into a capsule of the delivery system such that the capsule covers and constrains the valve prosthesis in a radially collapsed configuration, with the capsule extending over the annular groove of the piston and the attachment tabs received therein.

In an aspect of the third embodiment, and in combination with any other aspects herein, the disclosure provides that each attachment tab of the plurality of attachment tabs has an L-shaped configuration and includes a leg, a base, and a bend extending between the leg and the base, the base extending generally perpendicular to a longitudinal axis of the delivery system. The base of each attachment tab of the plurality of attachment tabs extends into the annular groove of the piston after the step of positioning the plurality of attachment tabs of the valve prosthesis into the annular groove of the piston of the delivery system.

In an aspect of the third embodiment, and in combination with any other aspects herein, the disclosure provides that the leg of each attachment tab of the plurality of attachment tabs extends generally parallel to the longitudinal axis of the delivery system after the step of positioning the plurality of attachment tabs of the valve prosthesis into the annular groove of the piston of the delivery system.

According to a fourth embodiment hereof, the disclosure provides a method of coupling a valve prosthesis to a delivery system for delivery thereof. A plurality of attachment tabs of the valve prosthesis are positioned into a plurality of circumferentially-extending grooves of a piston of the delivery system. The valve prosthesis includes a frame and a prosthetic valve disposed within the frame, the frame being self-expanding and including the plurality of attachment tabs extending from a first end of the frame. Each attachment tab of the plurality of attachment tabs has an L-shaped configuration and includes a leg, a base, and a bend extending between the leg and the base. The base has a first length and extends generally perpendicular to a longitudinal axis of the delivery system. The base has a first length and each circumferentially-extending groove has a length that is at least 300% greater than the first length. At least a portion of the valve prosthesis is positioned into a capsule of the delivery system such that the capsule covers and constrains the valve prosthesis in a radially collapsed configuration, with the capsule extending over the plurality of circumferentially-extending grooves of the piston and the attachment tabs received therein.

In an aspect of the fourth embodiment, and in combination with any other aspects herein, the disclosure provides that the base of each attachment tab of the plurality of attachment tabs extends into a circumferentially-extending groove of the plurality of circumferentially-extending grooves of the piston after the step of positioning the plurality of attachment tabs of the valve prosthesis into the plurality of circumferentially-extending grooves of the piston of the delivery system.

In an aspect of the fourth embodiment, and in combination with any other aspects herein, the disclosure provides that the leg of each attachment tab of the plurality of attachment tabs extends generally parallel to the longitudinal axis of the delivery system after the step of positioning the plurality of attachment tabs of the valve prosthesis into the plurality of circumferentially-extending grooves of the piston of the delivery system.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 depicts a perspective view of a prosthetic heart valve in accordance with an aspect of the disclosure.

FIG. 2 depicts a perspective view of a valve support of the prosthetic heart valve of FIG. 1 with a valve component secured therein in accordance with an aspect of the disclosure.

FIG. 3 depicts an atrial end view of the prosthetic heart valve shown in FIG. 1 in accordance with an aspect of the disclosure.

FIG. 4 depicts a ventricular end view of the prosthetic heart valve shown in FIG. 1 in accordance with an aspect of the disclosure.

FIG. 5 depicts a side view of a delivery system according to an embodiment hereof, wherein the delivery system is configured for delivering the prosthetic heart valve of FIG. 1 within a capsule of the delivery system.

FIG. 5A is a cross-sectional view taken along line A-A of FIG. 5.

FIG. 5B is a sectional view of FIG. 5.

FIG. 6 is an exploded view of the delivery system of FIG. 5.

FIG. 6A is an enlarged view of a distal portion of an innermost shaft assembly of the delivery system of FIG. 6, wherein the innermost shaft assembly includes a flexible shaft, a piston mount, a piston, a tension cable, a distal shaft, a capsule, and a capsule cap.

FIG. 6B is an enlarged view of a first subassembly of the innermost shaft assembly of FIG. 6A, wherein the first subassembly includes the flexible shaft, the piston mount and the piston.

FIG. 6C is an enlarged view of a second subassembly of the innermost shaft assembly of FIG. 6A, wherein the second subassembly includes the tension cable, the distal shaft, the capsule, and the capsule cap.

FIG. 6D is a sectional view of the distal shaft and the capsule cap of FIG. 6C.

FIG. 7 is a perspective view of a piston of the delivery system of FIG. 5, wherein the piston is shown removed from the delivery system for sake of illustration only.

FIG. 8 is a side view of a distal portion of the delivery system of FIG. 5, wherein the distal portion includes the capsule and the piston, the capsule being shown in a first position relative to the piston. The prosthetic heart valve of FIG. 1 is loaded into the capsule of the delivery system.

FIG. 9 is a side view of the distal portion of FIG. 7, the capsule being shown in a second position relative to the piston. The prosthetic heart valve of FIG. 1 is not shown for sake of illustration only.

FIG. 10 is a side view of the distal portion of FIG. 7, the capsule being shown in a third position relative to the piston. The prosthetic heart valve of FIG. 1 is not shown for sake of illustration only.

FIG. 11 is a perspective view of a piston of the delivery system of FIG. 5, wherein the piston is shown removed from the delivery system for sake of illustration only and attachment tabs of the prosthetic heart valve of FIG. 1 are coupled to the piston.

FIG. 11A is a side view of the piston of FIG. 11.

FIG. 12 is a side view of an outflow end of the prosthetic heart valve of FIG. 1 and the attachment tabs thereof.

FIG. 12A is an enlarged view of an attachment tab of FIG. 12, wherein the attachment tab has a L-shaped configuration.

FIG. 13 is a perspective view of a piston according to another embodiment hereof, wherein the piston is shown removed from the delivery system for sake of illustration only and the piston include three circumferentially-extending grooves for receiving the attachment tabs of the prosthetic heart valve of FIG. 1.

FIG. 14 is an end view of the piston of FIG. 13.

FIG. 15 is a perspective view of a piston according to another embodiment hereof, wherein the piston is shown removed from the delivery system for sake of illustration only and the piston include four circumferentially-extending grooves for receiving the attachment tabs of the prosthetic heart valve of FIG. 1.

FIG. 16 is an end view of the piston of FIG. 15.

FIG. 17 is an enlarged view of an attachment tab for the prosthetic heart valve of FIG. 1 according to another embodiment hereof, wherein the attachment tab has a curved configuration.

FIG. 18 is an enlarged view of an attachment tab for the prosthetic heart valve of FIG. 1 according to another embodiment hereof, wherein the attachment tab has a curved configuration and a spherical tip.

FIG. 19 is an enlarged view of an attachment tab for the prosthetic heart valve of FIG. 1 according to another embodiment hereof, wherein the attachment tab has an angled configuration.

FIG. 20 is a schematic side view of loading the prosthetic heart valve of FIG. 1 into a capsule of the delivery system of FIG. 5, according to an embodiment hereof.

FIG. 20A is a schematic side view of loading a prosthetic heart valve having T-shaped attachment tabs into a capsule via a piston with T-shaped recesses.

FIG. 20B is perspective view of the piston of FIG. 20A.

FIGS. 21-24 illustrate a method of delivering the prosthetic heart valve of FIG. 1 to a native mitral valve annulus with the delivery system of FIG. 5, according to an embodiment hereof.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. With respect to a prosthetic valve device, the terms “proximal” and “distal” can refer to the location of portions of the device with respect to the direction of blood flow. For example, proximal can refer to an upstream position or a location where blood flows into the device (e.g., inflow region), and distal can refer to a downstream position or a location where blood flows out of the device (e.g., outflow region).

As referred to herein, implants, prostheses, prosthetic heart valves or prosthetic valves useful with the various systems, devices and methods of the present disclosure may assume a wide variety of configurations. Prosthetic heart valves can include, for example, a bioprosthetic heart valve having tissue leaflets or a synthetic heart valve having polymeric, metallic or tissue-engineered leaflets, and can be specifically configured for replacing valves of the human heart. The prosthetic valves of the present disclosure may be self-expandable, balloon expandable and/or mechanically expandable or combinations thereof. In general terms, the prosthetic valves of the present disclosure include a stent or stent frame having an internal lumen maintaining a valve structure (tissue or synthetic), with the stent frame having a normal, expanded condition or arrangement and collapsible to a compressed condition or arrangement for loading within the delivery system. For example, the stents or stent frames are support structures that comprise a number of struts or wire segments arranged relative to each other to provide a desired compressibility and strength to the prosthetic valve. The struts or wire segments are arranged such that they are capable of self-transitioning from, or being forced from, a compressed or collapsed arrangement to a normal, radially expanded arrangement. The struts or wire segments can be formed from a shape memory material, such as a nickel titanium alloy (e.g., Nitinol). The stent frame can be laser-cut from a single piece of material, or can be assembled from a number of discrete components.

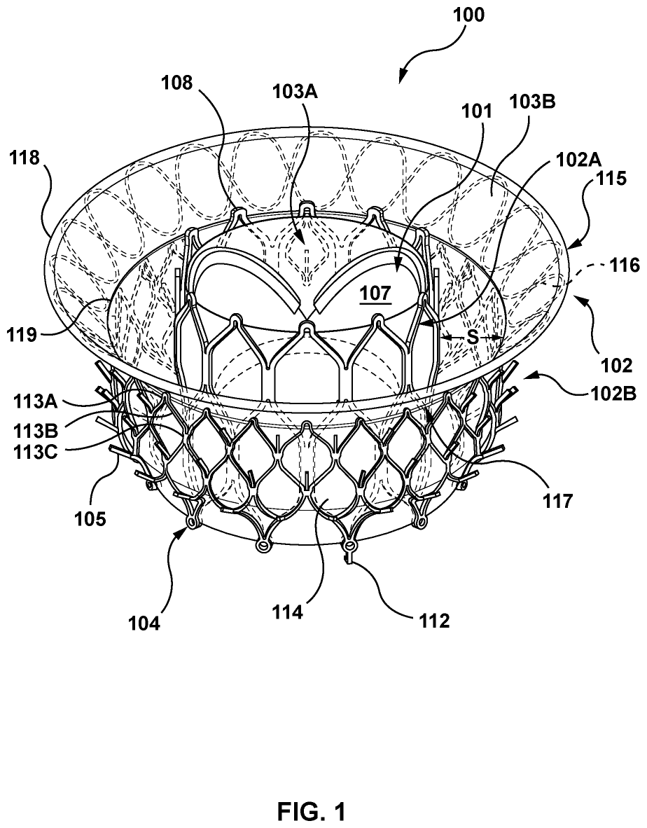

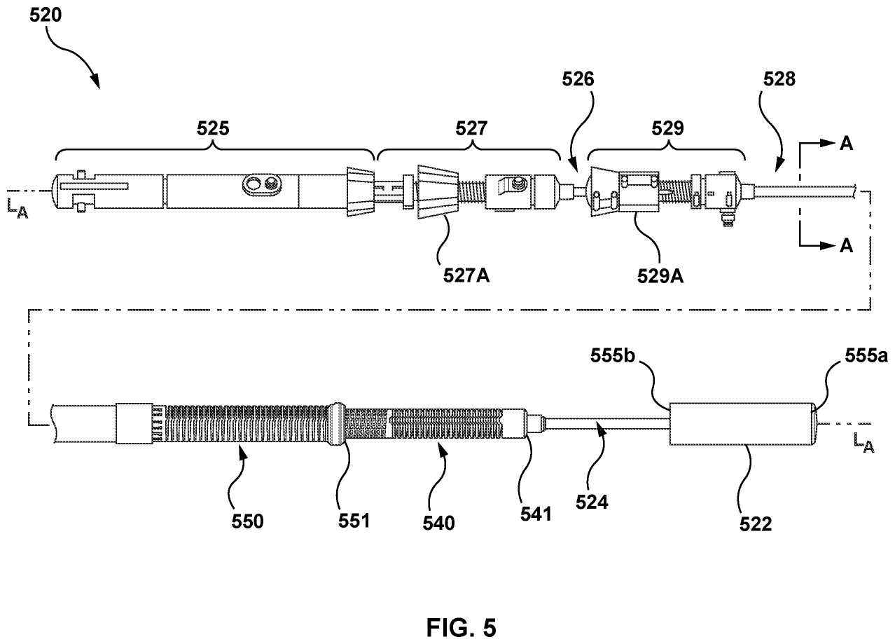

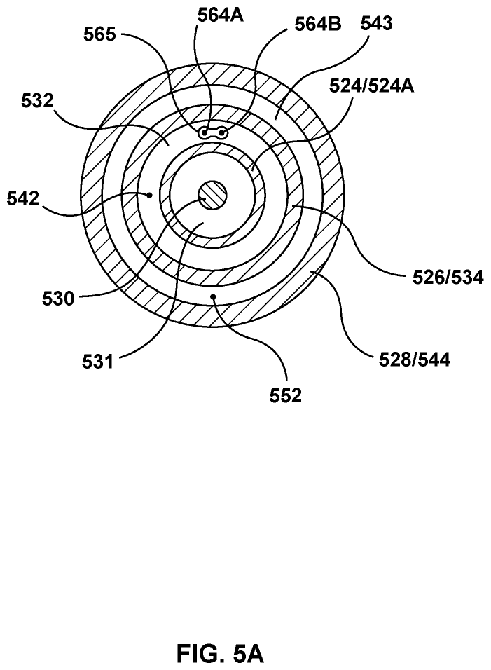

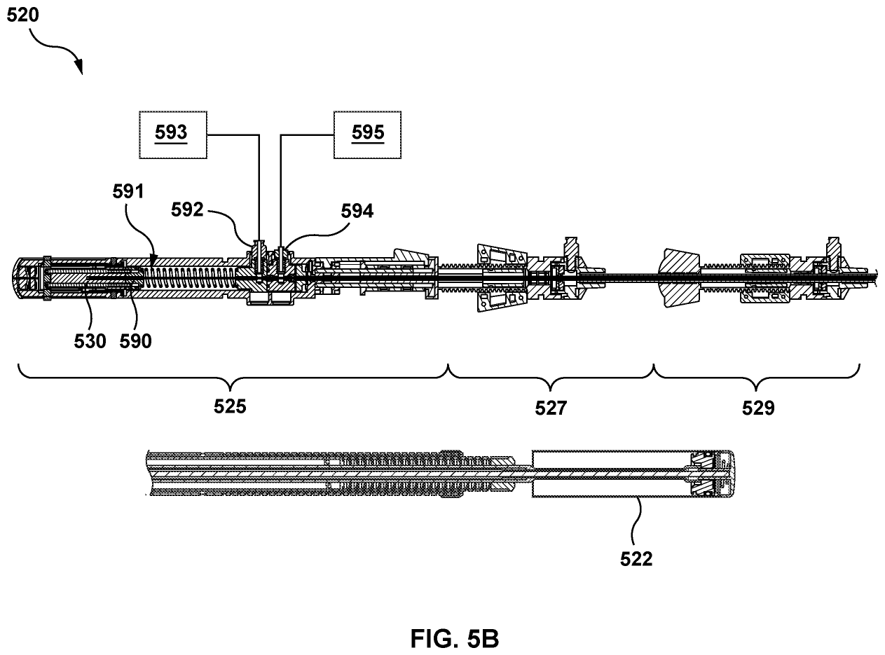

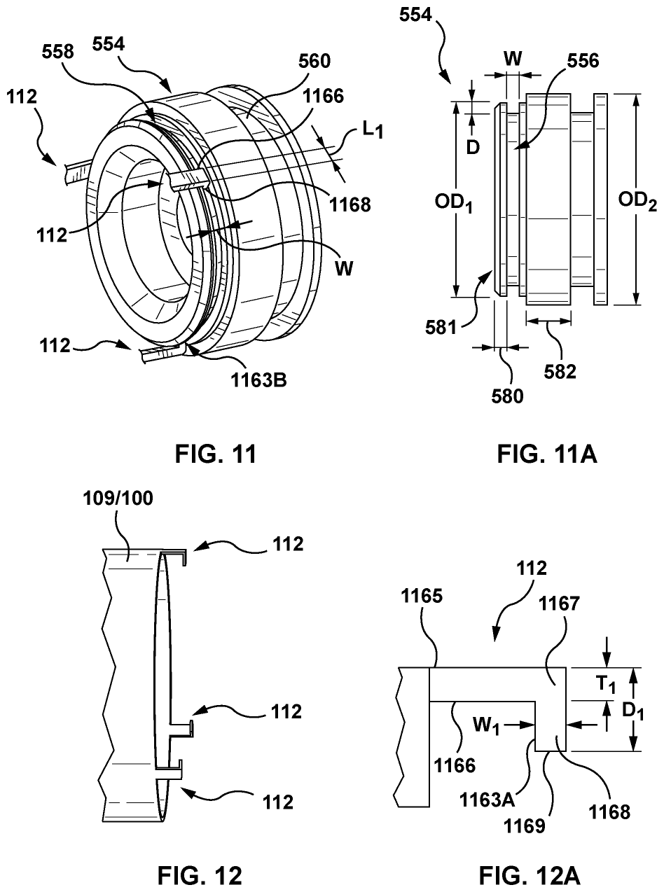

Systems and methods of the disclosure include a delivery system having a capsule for radially compressing a prosthetic heart valve. In embodiments hereof, the prosthetic heart valve is coupled to the delivery system via a piston. The piston includes an annular groove, or a plurality of circumferentially-extending grooves, for receiving attachment tabs of the prosthetic heart valve. The detailed description hereof first includes a description of an exemplary prosthetic heart valve in FIGS. 1-4 that may be used in embodiments hereof, and further includes a description of a delivery system in FIGS. 5-10 that may be used in embodiments hereof. The coupling between the prosthetic heart valve and the delivery system, in which the attachment tabs are loaded into the annular groove or circumferentially-extending grooves of the piston according to various embodiments hereof, is further described in FIGS. 11-19. It will be understood by one of ordinary skill in the art that the prosthetic heart valve of FIGS. 1-4 and the delivery system of FIGS. 5-10 are exemplary, and alternative configurations thereof may be utilized in accordance with the principles described herein.

FIGS. 1-4 illustrate an exemplary prosthetic heart valve 100 for use in embodiments hereof. Prosthetic heart valve 100 is illustrated herein in order to facilitate description of the interaction between the prosthetic heart valve 100 and a delivery system to be utilized in conjunction therewith according to embodiments hereof. It is understood that any number of alternate heart valve prostheses can be used with the methods and devices described herein. The prosthetic heart valve 100 is presented by way of example only, and other shapes and designs of prosthetic heart valves are also consistent with embodiments hereof. Other non-limiting examples of prosthetic heart valves that can be delivered via the delivery systems and methods described herein are described in U.S. application Ser. No. 16/853,851 to McVeigh et al., U.S. Pat. No. 9,034,032 to McLean et al. and International Patent Application No. PCT/US21724/029549 to McLean et al, each of which is incorporated by reference herein in its entirety. Although the prosthetic heart valve 100 is configured for placement within a mitral heart valve or a tricuspid heart valve, embodiments of delivery systems and techniques described herein may be used in conjunction with any transcatheter valve prostheses. For example, embodiments described herein may be utilized with a transcatheter prosthetic heart valve configured for placement within a pulmonary, aortic, mitral, or tricuspid valve. There is no intention to be bound by any expressed or implied theory presented in the preceding technical field, background, brief summary or the following detailed description.

The prosthetic heart valve 100 is configured to be radially compressed into a reduced-diameter configuration (not shown) for delivery within a vasculature and to return to an expanded, deployed configuration, which is shown in FIGS. 1-4. Stated another way, the prosthetic heart valve 100 has a crimped configuration for delivery within a vasculature and an expanded configuration for deployment within a native heart valve. The prosthetic heart valve 100 may also be implanted in a previously implanted valve (either transcatheter or surgically). In accordance with embodiments hereof, when in the radially compressed or reduced-diameter configuration, the prosthetic heart valve 100 has a low profile suitable for delivery to and deployment within a native heart valve via a suitable delivery system that may be tracked to the deployment site of the native heart valve of a heart via any one of a transatrial, antegrade, or transapical approach. The prosthetic heart valve 100 includes a stent or frame 102 and a valve component 101 including at least one leaflet 107 disposed within and secured to the frame 102. The valve component 101 of the prosthetic heart valve 100 is capable of regulating flow therethrough via valve leaflets that may form a replacement valve.

Any portion of the frame 102 described herein as an element of a heart valve prothesis 100 may be made from any number of suitable biocompatible materials, e.g., stainless steel, nickel titanium alloys such as Nitinol™, cobalt chromium alloys such as MP35N, other alloys such as ELGILOY@(Elgin, Ill.), various polymers, pyrolytic carbon, silicone, polytetrafluoroethylene (PTFE), or any number of other materials or combination of materials. A suitable biocompatible material would be selected to provide the transcatheter heart valve prothesis 100 to be configured to be compressed into a reduced-diameter crimped configuration for transcatheter delivery to a native valve, whereby release from a delivery catheter returns the prosthesis to an expanded, deployed configuration. Alternatively, the prosthetic heart valve 100 may be balloon-expandable as would be understood by one of ordinary skill in the art.

In an aspect of the disclosure, the frame 102 of the prosthetic heart valve 100 includes a valve support 102A at least partially surrounded by and attached to an anchoring member 102B. The valve support 102A is configured to support the valve component 101 therein. The valve support 102A is a tubular stent-like or frame structure that defines a central lumen from a first end 108 of the valve support 102A to a second end 109 of the valve support 102A. When positioned in situ within a native triscupid valve, the first end 108 is an inflow or upstream end and the second end 109 is an outflow or downstream end. At the outflow end 109, the valve support 102A is attached to the anchoring member 102B via a plurality of connector components 104. In an embodiment, the plurality of connector components are rivets.

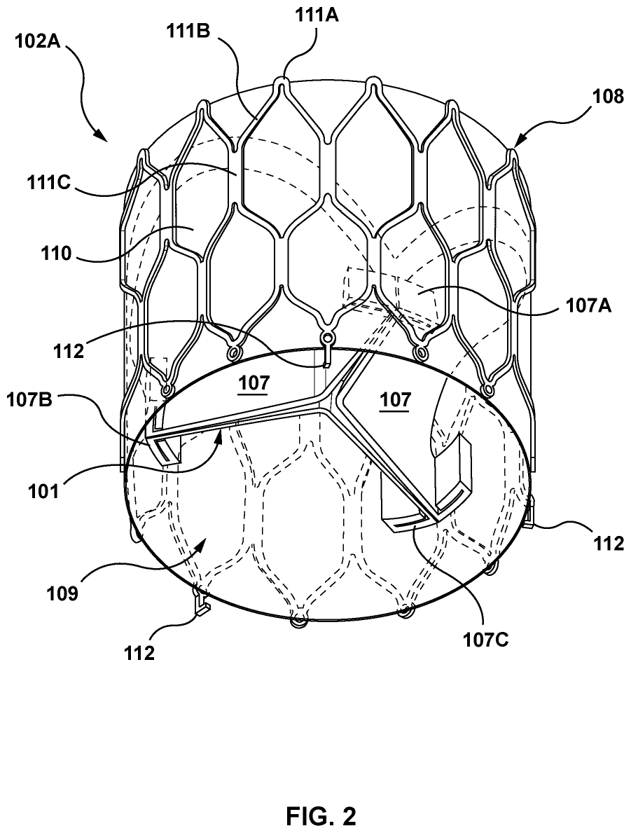

Referring to FIG. 2, the structure of the valve support 102A will now be described in more detail. The valve support 102A includes a plurality of crowns 111A and a plurality of struts 111B with each crown 111A being formed between a pair of opposing struts 111B. Each crown 111A is a curved segment or bend extending between opposing struts 111B. The valve support 102A is tubular, with a plurality of side openings 110 being defined by edges of the plurality of crowns 111A and the plurality of struts 111B. In an embodiment, the plurality of side openings 110 may be substantially diamond-shaped. The valve support 102A includes a plurality of nodes 111C. A node 111C is defined as a region where two crowns of the plurality of crowns 111A within the valve support 102A meet or connect. At the outflow end 109 thereof, the valve support 102A includes a plurality of attachment tabs 112 extending therefrom that function to releasably couple the prosthetic heart valve 100 to a delivery system. In an embodiment, the valve support 102A includes exactly three attachment tabs 112 that are circumferentially spaced apart from each other at equal intervals.

The anchoring member 102B is a stent-like or frame structure that functions as an anchor for the prosthetic heart valve 100 to secure its deployed position within a native annulus. The anchoring member 102B is a substantially cylindrically-shaped structure that is configured to engage heart tissue at or below an annulus of a native heart valve, such as an annulus of a native mitral valve. At the inflow end 108 of the valve support 102A, the anchoring member 102B is radially spaced a distance S from the valve support 102A to mechanically isolate the inflow end 108 of the valve support 102A from the anchoring member 102B. The anchoring member 102B includes one or more fixation elements 105 that extend outward from an exterior side thereof to engage heart tissue. The fixation elements 105 project radially outward and are inclined toward an upstream direction. The fixation elements 105, for example, can be prongs, cleats, barbs, hooks, or other elements that are inclined only in the upstream direction (e.g., a direction extending away from the downstream portion of the prosthetic heart valve 100. In an embodiment, the anchoring member 102 includes exactly three rows of fixation elements 105.

The anchoring member 102B includes a plurality of crowns 113A and a plurality of struts 113B with each crown 113A being formed between a pair of opposing struts 113B. Each crown 113A is a curved segment or bend extending between opposing struts 113B. The anchoring member 102B is tubular, with the plurality of side openings 114 being defined by edges of the plurality of crowns 113A and the plurality of struts 113B. In an embodiment, the plurality of side openings 114 may be substantially diamond-shaped. The anchoring member 102B includes a plurality of nodes 113C. A node 113C is defined as a region where two crowns of the plurality of crowns 113A within the anchoring member 102B meet or connect. When attached to the valve support 102A via the plurality of connecting components 104, the anchoring member 102B forms an outer frame portion of the frame 102 and the valve support 102A forms an inner frame portion of the frame 102 with the anchoring member 102B circumferentially surrounding the valve support 102 disposed therein.

Each of the valve support 102A and the anchoring member 102B include a skirt or graft material 103A, 103B, respectively, secured thereto. More particularly, the graft material 103A is coupled to an inner surface of the valve support 102A to line a portion thereof. Alternatively, the graft material 103A may be coupled to an outer surface of the valve support 102A to enclose a portion thereof as would be known to one of ordinary skill in the art of prosthetic valve construction. The graft material 103B is coupled to an inner surface of the anchoring member 102B to line a portion thereof. The outer engagement surface of the anchoring member 102 is not covered by any sealing or graft material so that the outer engagement surface directly contacts the tissue of the native annulus. The graft material 103A, 103B may be a natural or biological material such as pericardium or another membranous tissue such as intestinal submucosa. Alternatively, the graft material 103A, 103B may be a low-porosity woven fabric, such as polyester, Dacron fabric, or PTFE, which creates a one-way fluid passage when attached to the stent.

The prosthetic heart valve 100 further includes an extension member or brim 115 that extends outwardly from an inflow end of the anchoring member 102B. The brim 115 is formed by a brim support 116 and a flexible web, which in this embodiment is a portion of graft material 103B that extends past or beyond the inflow end of the anchor 102B. More particularly, the graft material 103B (which is coupled to an inner surface of the anchoring member 102B as described above) extends past or beyond the inflow end of the anchoring member 102B, and includes an integral folded pocket or hem beyond the inflow end of the anchoring member 102B. The brim support 116 is disposed within this folded pocket of the graft material 103B. In the depicted embodiment, the brim support 116 includes overlapping, 180 degree out of phase sinusoidal wire forms. However, the brim support 116 may have other configurations. The brim 115 may act as an atrial retainer, if present, and to serve such a function the brim 115 may be configured to engage tissue above a native annulus, such as a supra-annular surface or some other tissue in the right atrium, to thereby inhibit downstream migration of a prosthetic heart valve 100. Accordingly, the brim 115 is of a larger diameter than the frame 102 and extends radially outward from the anchoring member 102B. The portion of graft material 103B connecting the brim 115 to the anchoring member 102B is referred to herein as a brim hinge 117. The brim hinge 117 is configured to permit the brim 115 to hinge and/or flex with respect to the remainder of the prosthetic heart valve 100.

In the depicted embodiment, the brim 115 includes an extension or continuation of the graft material 103B as described above. There is no metal-to-metal connection between the anchoring member 102B and the brim support 116. Thus, the brim 115 is a floppy structure that can readily flex with respect to the anchoring member 102B. However, it is not required that the brim 115 includes an extension or continuation of the graft material 103B. In another embodiment, the brim 115 is a separate component including a flexible web (e.g., graft material or fabric) and the brim support 116 attached thereto, and the brim is attached to the graft material 103B and/or an inflow end of the anchoring member 102B. The brim 115 includes a first end or edge 118 and a second end or edge 119.

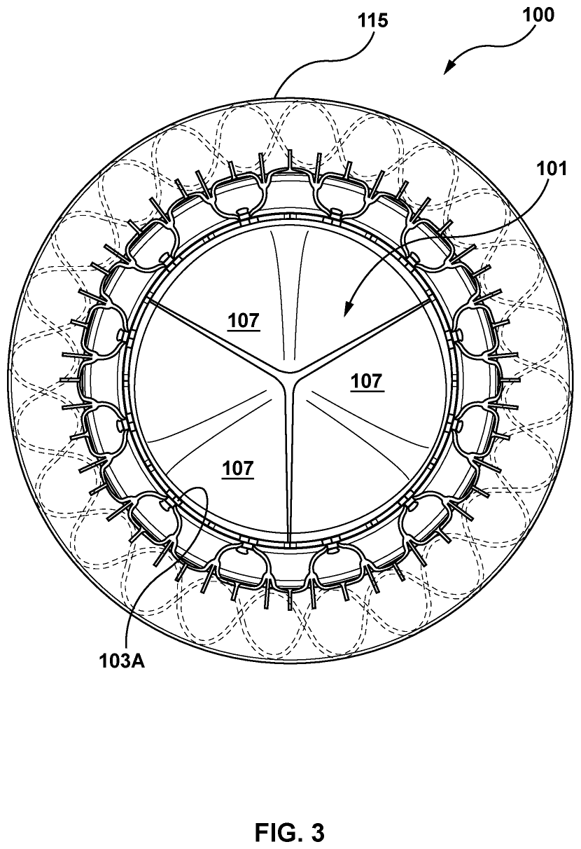

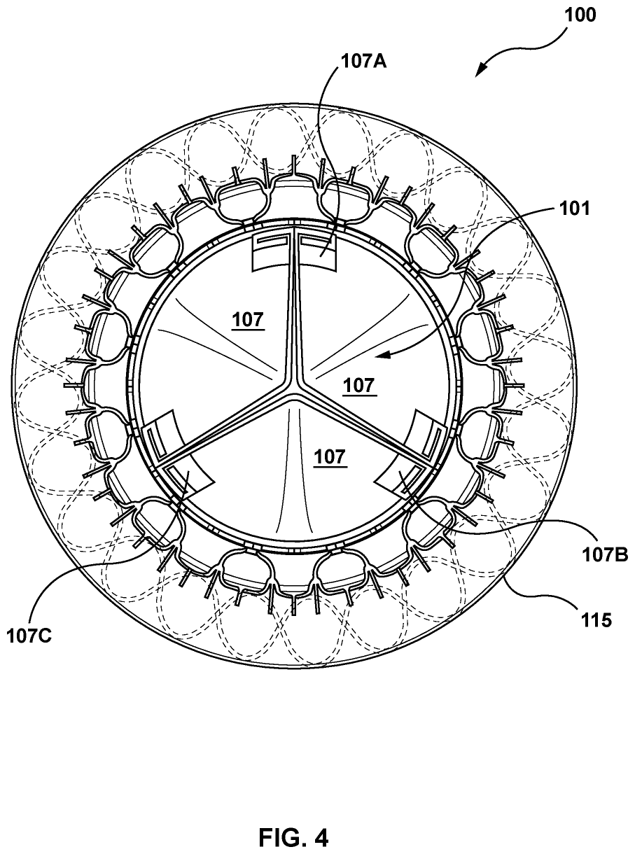

The valve component 101 of the prosthetic heart valve 100 is capable of regulating flow therethrough via valve leaflets 107 that may form a replacement valve. FIGS. 1-4 illustrate an exemplary valve component having three leaflets, although a single leaflet or bicuspid leaflet configuration may alternatively be used in embodiments hereof. When deployed in situ, the valve component 101 in a closed state is configured to block blood flow in one direction to regulate blood flow through the central lumen of the valve support 102A. FIG. 2 depicts a perspective view of the valve support 102A with a valve component 101 secured therein, the valve support 102A being shown in FIG. 2 removed from the remainder of the prosthetic heart valve 100 shown in FIG. 1 for ease of illustration. FIG. 3 depicts an atrial or inflow end view of the prosthetic heart valve 100 shown in FIG. 1, and FIG. 4 depicts a ventricular or outflow end view of the prosthetic heart valve 100 shown in FIG. 1. The valve component 101 includes valve leaflets 107, e.g., three valve leaflets 107, that are disposed to coapt within an upstream portion of the valve support 102A with leaflet commissures 107A, 107B, 107C of the valve leaflets 107 being secured within a downstream portion of the valve support 102A, such that the valve leaflets 107 open during diastole. Leaflets 107 are attached along their bases to the valve support 102A, for example, using sutures or a suitable biocompatible adhesive. Adjoining pairs of leaflets 107 are attached to one another at their lateral ends to form leaflet commissures 107A, 107B, 107C. The orientation of the leaflets 107 within the valve support 102A depends upon on which end of the prosthetic heart valve 100 is the inflow end and which end of the prosthetic heart valve 100 is the outflow end, thereby ensuring one-way flow of blood through the prosthetic heart valve 100.

The valve leaflets 107 are attached to the graft material 103A in order to form the valve component 101. The valve leaflets 107 may be formed of various flexible materials including, but not limited to natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

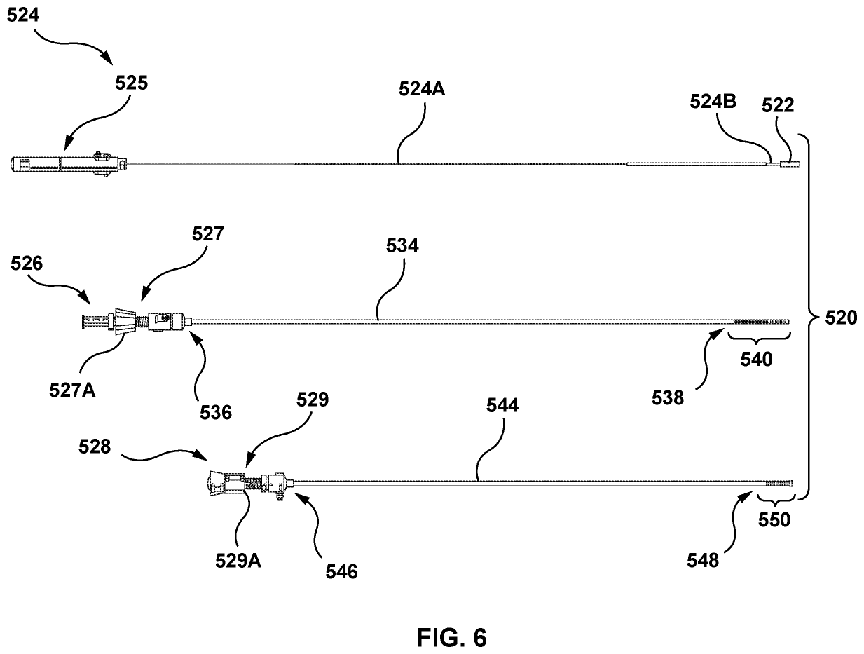

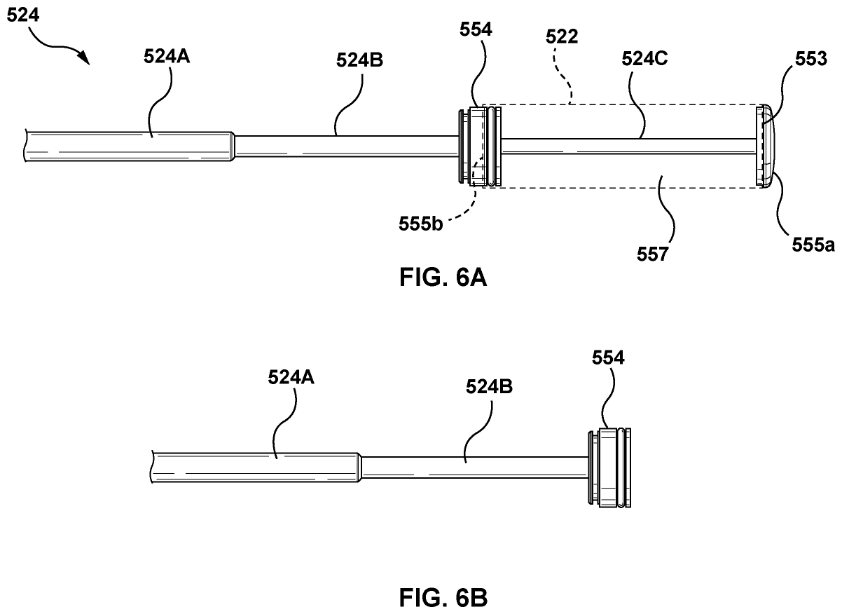

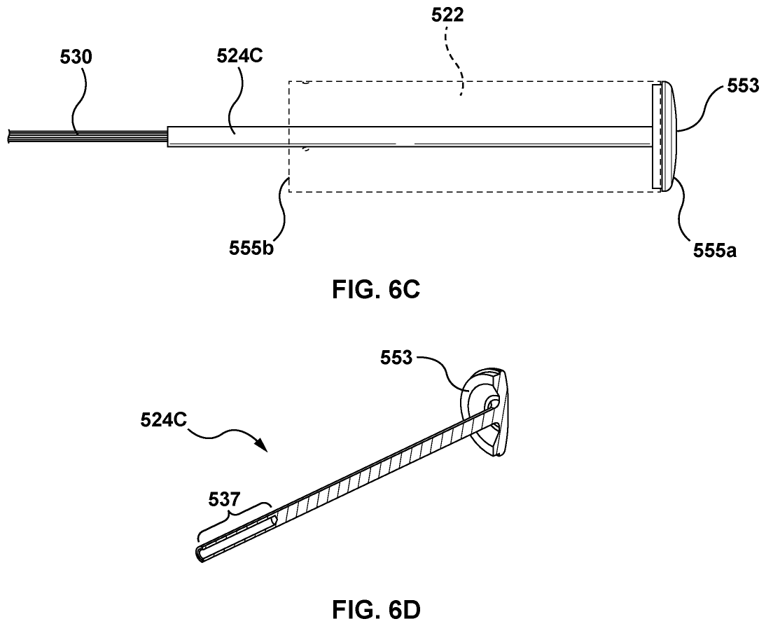

A delivery system 520 which may be used for transcatheter delivery and deployment of an implant, such as the non-limiting example of the prosthetic heart valve 100 of FIGS. 1-4, is shown in FIGS. 5-6D. In general terms, the delivery system 520 is arranged and configured for percutaneously delivering an implant (e.g., prosthetic heart valve 100) in a delivery configuration to a patient's native defective heart valve or other portion of a patient's anatomy via transcatheter delivery. FIG. 5 illustrates a side view of the delivery system 520, and FIG. 5A is a cross-sectional view taken along line A-A of FIG. 5. FIG. 6 is an exploded view of the delivery system 520. The delivery system 520 includes a capsule 522 for housing at least a portion of the prosthetic heart valve 100, an innermost shaft assembly 524 contained within and coupled to the capsule 522, an inner steerable catheter 526 disposed over the innermost shaft assembly 524, and an outer steerable catheter 528 disposed over the inner steerable catheter 526. The inner steerable catheter 526 includes a handle 527 at a proximal portion thereof for manipulation in situ, and the outer steerable catheter 528 includes a handle 529 at a proximal portion thereof for manipulation in situ. During delivery, the prosthetic heart valve 100 contained within the capsule 522 is steered by the inner steerable catheter 526 and the outer steerable catheter 528 into alignment within the native heart valve for which the prosthetic heart valve 100 serves as a replacement. The inner steerable catheter 526 may be controlled or steered independently from the outer steerable catheter 528, and provides the delivery system 520 with omnidirectional steering capabilities to direct the capsule 522.

Components of the delivery system 520 will now be described in more detail. At a proximal end thereof, as best shown in the exploded view of FIG. 6, the innermost shaft assembly 524 is fixedly secured to a manifold 525. FIG. 6A is an enlarged view of a distal portion of the innermost shaft assembly 524. The innermost shaft assembly 524 includes a flexible shaft 524A, a piston mount 524B, a piston 554, a tension cable 530, a distal shaft 524C, a capsule 522, and a capsule cap 553. The innermost shaft assembly 524 may be considered to include a first subassembly, shown in FIG. 6B and which includes the flexible shaft 524A, the piston mount 524B and the piston 524, and a second subassembly, shown in FIG. 6C and which includes the tension cable 530, the distal shaft 524C, the capsule 522, and the capsule cap 533. The first and second subassemblies are coupled together in that the distal shaft 524C of the second subassembly slides or telescopes within the piston mount 524B of the first subassembly. In addition, the first and second subassemblies are coupled together via the manifold 525.

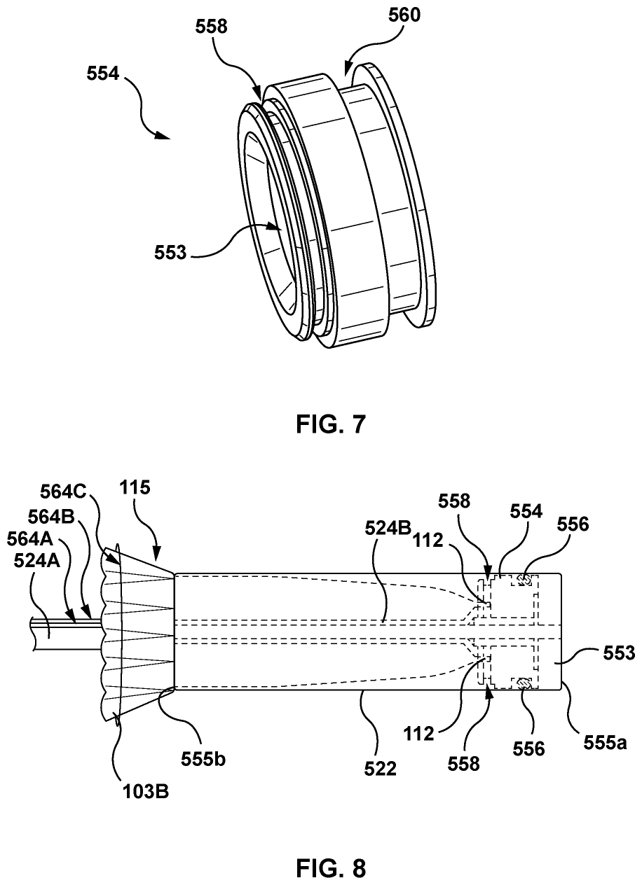

With respect to the first subassembly shown in FIG. 6B, the flexible shaft 524A is a flexible elongated tubular body that may include, for example, a flexible metal spring disposed within a polymer jacket. A distal end of the flexible shaft 524A is attached and fixed relative to a proximal end of the piston mount 524B, which is a rigid, tubular body that distally extends from the flexible shaft 524A. The piston 554 is attached and fixed relative to the piston mount 524B. As will be explained in more detail herein with respect to FIG. 7, the piston 554 is disposed over and mounted to a distal end of the piston mount 524B.

With respect to the second subassembly shown in FIG. 6C, the capsule 522 is a tubular component having a closed or distal end 555a and an open or proximal end 555b. The capsule 522 may be rigid and made of metal. As will be described in more detail herein with respect to FIGS. 7-10, the capsule 522 capsule 522 is configured to house at least a portion of the prosthetic heart valve 100 during delivery. The distal end 555a of the capsule 522 is closed via a capsule cap 553. The capsule cap 553 may be integrally formed with the capsule 522 or may be a separate component attached thereto to form the closed distal end 555a. The distal shaft 524C is further attached to the capsule cap 553. FIG. 6D is a sectional view of the distal shaft 524C and the capsule cap 553. As shown in FIG. 6D, the distal shaft 524C may be integrally formed with the capsule cap 553, or in another embodiment, the distal end of the distal shaft 524C may be welded or otherwise attached to the capsule cap 553.

With additional reference to the cross-sectional view of FIG. 5A and the sectional view of FIG. 5B, the tension cable 530 extends from the manifold 525 to the distal shaft 524C through the lumens of the flexible shaft 524A and the piston mount 524B. The lumens of the flexible shaft 524A and the piston mount 524B are in fluid communication with each other, and are designated with the reference number 531 on FIG. 5A. A distal end of the tension cable 530 is secured or mounted within a proximal portion 537 of the distal shaft 524C. In an embodiment, the tension cable 530 is configured to be selectively tensioned (proximally or distally) by hydraulic pressure to enable translation of the capsule 522 either proximally or distally with respect to the piston mount 524B and the piston 554 attached thereto. Depending on which hydraulic system is engaged (deployment or recapture), the tension cable 530 will translate under tension through the innermost shaft assembly 524, with the capsule 522 moving distally during deployment or proximally during recapture.

More particularly, as best shown in FIG. 5B, the manifold 525 includes a recapture piston 590 and the recapture chamber 591. The recapture chamber 591 is disposed within the manifold 525 and is configured to house the recapture piston 590 therein. The manifold also includes a deployment valve 594 that is configured to be coupled to a first external source or pump 595 of hydraulic fluid and a recapture valve 592 that is configured to be coupled to a second external source or pump 593 of hydraulic fluid. Hydraulic fluid is delivered from the pump 595 through the deployment valve 594 in order to drive the capsule 522 in a distal direction, thereby deploying the prosthetic heart valve 100, as will be described in more detail herein with respect to FIGS. 8-10. Hydraulic fluid is delivered to from the pump 593 through the recapture valve 592 of the delivery device 320 in order to drive the capsule 522 in a proximal direction, thereby recapturing the prosthetic heart valve 100. More particularly, the recapture chamber 591 is fluidly coupled to the recapture valve 592 and is configured to fill with hydraulic fluid. As the hydraulic fluid flows into and/or exits from the recapture chamber 591, the recapture piston 590 is configured to move axially within the recapture chamber 591.

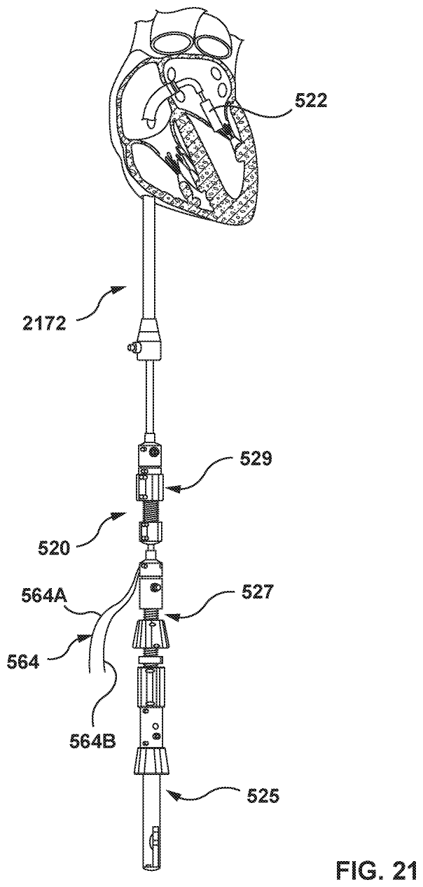

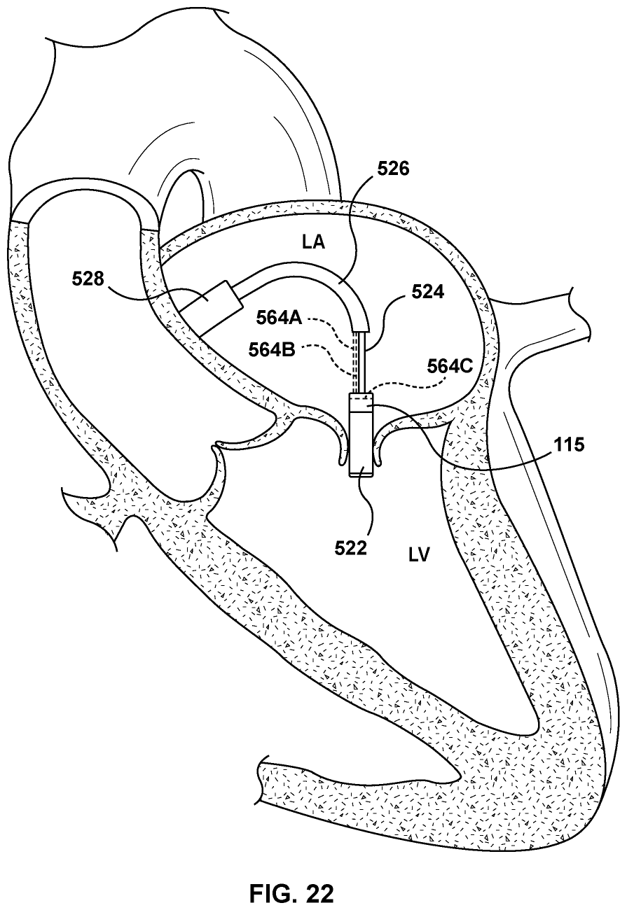

The recapture piston 590 is operably coupled to the capsule 522 via the tension cable 530, meaning that movement of either will place an axial force onto the opposing component. More particularly, a proximal end of the tension cable 530 is attached to the recapture piston 590 and a distal end of the tension cable 530 is attached the distal shaft 524C, which is, subsequently coupled to the capsule cap 553 as described above. The tension cable 530 remains taut or under tension between the recapture piston 590 and the distal shaft 524C, resulting in a force that encourages the recapture piston 590 and distal shaft 524C to move together. Prior to deployment of the prosthetic heart valve 100, the recapture piston 590 is disposed at a proximal end of the recapture chamber 590 and the recapture chamber 591 is filled with hydraulic fluid. As the capsule 522 is driven distally as described in more detail with reference to FIGS. 7-10, the recapture piston 590 is also pulled distally since the proximal end of the tension cable is attached thereto and hydraulic fluid within the recapture chamber 591 is pushed out of the recapture chamber 591 as the recapture piston 590 is pulled distally during deployment of the prosthetic heart valve 100. However, it may be desired to recapture the prosthetic heart valve 100 after deployment thereof as described in more detail with respect to FIGS. 21A-21D. During recapture, hydraulic fluid is delivered to from the pump 593 through the recapture valve 592 of the delivery device 320 in order to drive the capsule 522 in a proximal direction, thereby recapturing the prosthetic heart valve 100.

The distal shaft 524C is received within the lumen of the piston mount 524B and may move or slide relative thereto in an axial or longitudinal direction. Stated another way, the distal shaft 524C telescopes within the piston mount 524B. The capsule 522 is concentrically disposed over the distal shaft 524C, and an annular chamber 557 (shown in FIG. 6A) is defined between an inner surface of the capsule 522, an outer surface of the distal shaft 524C, the piston 554 and the capsule cap 553. As will be described in more detail with respect to FIGS. 7-10, the annular chamber 557 is part of a hydraulic deployment system that is configured to cause proximal and distal translation of the capsule 522 with respect to the prosthetic heart valve 100 for deployment thereof. The annular chamber 557 is a sealed cavity formed by the piston 554, the seal 556, the capsule 552, and the capsule cap 553 into which fluid can be introduced to increase fluid pressure therein and thereby move the capsule 522 away from the piston 554, which remains stationary during fluid delivery as described below.

The inner steerable catheter 526 is disposed over the innermost shaft assembly 524 such that an annular lumen 532 (shown on FIG. 5A) is defined between an outer surface of the innermost shaft assembly 524 and an inner surface of the inner steerable catheter 526 along an entire length of the inner steerable catheter 526. The innermost shaft assembly 524 is slidingly disposed within the inner steerable catheter 526 such that relative axial movement is permitted therebetween as will be described in more detail below. As used herein, “slidably” generally denotes back and forth movement in a longitudinal direction along or generally parallel to a central longitudinal axis L_{A} of the delivery system 520. The inner steerable catheter 526 includes a flexible, steerable tubular component or shaft 534, the handle 527 fixedly secured to a proximal end 536 of the shaft 534, an inner distal flex component 540 extending distally from a distal end 538 of the shaft 534, and a first pull-wire 542. The shaft 534 can assume various forms conventionally employed, and in some embodiments can be a braided catheter surrounded by a polymer outer layer or jacket. The inner distal flex component 540 is secured to and extends distally from the shaft 534, and can be configured to exhibit flexibility and/or hoop strength characteristics differing from that of the shaft 534. In an embodiment, the inner distal flex component 540 is a metal tube with a laser cut pattern that facilitates flexing or bending of the inner distal flex component 540. A cap 541 is attached to a distal end of the inner distal flex component 540. The cap 541 is an annular component that permits the innermost shaft assembly 524 to slide therethrough. The handle 527 includes an actuator 527A that is accessible to the user and may be manipulated to control flexing or bending of the inner distal flex component 540 of the shaft 534. More particularly, as will be explained in more detail herein, the first pull-wire 542 is attached to and extends between the handle 527 and the cap 541 attached to the inner distal flex component 540. The first pull-wire 542 is selectively tensioned by the user to bend the inner distal flex component 540. The inner steerable catheter 526 is configured to transition between a non-flexed configuration when the first pull-wire 542 is not tensioned and a flexed configuration in which the first pull-wire 542 is tensioned.

The handle 527 includes the actuator 527A for tensioning the first pull-wire 542. The handle 527 can have any shape or size appropriate for convenient handling by a user. The actuator 527A is coupled to the proximal end of the first pull-wire 542, and is generally constructed to provide selective proximal retraction and distal advancement of the first pull-wire 542. Stated another way, the actuator 527A is coupled to the proximal end of the first pull-wire 542 and is constructed to selectively push or pull the first pull-wire 542. The actuator 527A may assume any construction that is capable of providing the desired pull-wire actuation functionality. In an embodiment, the actuator 527A is configured as a rotatable knob that is rotated in a first direction (i.e., clockwise) to proximally retract the first pull-wire 542 and apply tension thereto, and is rotated in a second, opposing direction (i.e., counter-clockwise) to distally advance the first pull-wire 542 and remove or release tension therefrom, such as the rotatable knob described in U.S. Pat. No. 10,188,833 to Bolduc et al., filed Dec. 8, 2015, or the rotatable knob described in U.S. Pat. No. 6,607,496 to Poor et al., filed on Sep. 12, 2000, each of which is assigned to the same assignee as the present disclosure and which is herein incorporated by reference in its entirety. In another embodiment, the actuator 527A may be configured as a button such as those described in U.S. Pat. No. 10,278,852 to Griffin, filed on Mar. 10, 2016, which is assigned to the same assignee as the present disclosure and which is herein incorporated by reference in its entirety.

The outer steerable catheter 528 is slidably disposed over the inner steerable catheter 526 such that an annular lumen 543 (shown on FIG. 5A) is defined between an outer surface of the inner steerable catheter 526 and an inner surface of the outer steerable catheter 528 along an entire length of the outer steerable catheter 528. The outer steerable catheter 528 includes a flexible, steerable tubular component or shaft 544, the handle 529 fixedly secured relative to a proximal end 546 of the shaft 544, an outer distal flex component 550 extending distally from a distal end 548 of the shaft 544, and a second pull-wire 552. The shaft 544 can assume various forms conventionally employed, and in some embodiments can be a braided catheter surrounded by a polymer outer layer or jacket. The outer distal flex component 550 is secured to and extends distally from the shaft 544, and can be configured to exhibit flexibility and/or hoop strength characteristics differing from that of the shaft 544. In an embodiment, the outer distal flex component 550 is formed from a metal tube with a laser cut pattern that facilitates flexing or bending of the outer distal flex component 550. A cap 551 is attached to a distal end of the outer distal flex component 550. The cap 551 is an annular component that permits the inner steerable catheter 526 to slide therethrough. The handle 529 includes an actuator 529A that is accessible to the user and may be manipulated to control steering of the outer distal flex component 550 of the shaft 544. More particularly, as will be explained in more detail herein, the second pull-wire 552 is attached to and extends between the handle 529 and the cap 551 of the outer distal flex component 550. The second pull-wire 552 is selectively tensioned by the user to bend the outer distal flex component 550. The outer steerable catheter 528 is configured to transition between a non-flexed configuration when the second pull-wire 552 is not tensioned and a flexed configuration in which the second pull-wire 552 is tensioned.

The handle 529 includes the actuator 529A for tensioning the second pull-wire 552. The handle 529 can have any shape or size appropriate for convenient handling by a user. The actuator 529A is coupled to the proximal end of the second pull-wire 552, and is generally constructed to provide selective proximal retraction and distal advancement of the second pull-wire 552. Stated another way, the actuator 529A is coupled to the proximal end of the second pull-wire 552 and is constructed to selectively push or pull the second pull-wire 552. The actuator 529A may assume any construction that is capable of providing the desired pull-wire actuation functionality. In an embodiment, the actuator 529A is configured as a rotatable knob that is rotated in a first direction (i.e., clockwise) to proximally retract the second pull-wire 552 and apply tension thereto, and is rotated in a second, opposing direction (i.e., counter-clockwise) to distally advance the second pull-wire 552 and remove or release tension therefrom, such as the rotatable knob described in U.S. Pat. No. 10,188,833 to Bolduc et al., filed Dec. 8, 2015, or the rotatable knob described in U.S. Pat. No. 6,607,496 to Poor et al., filed on Sep. 12, 2000, each of which is assigned to the same assignee as the present disclosure and which is herein incorporated by reference in its entirety. In another embodiment, the actuator 529A may be configured as a button such as those described in U.S. Pat. No. 10,278,852 to Griffin, filed on Mar. 10, 2016, which is assigned to the same assignee as the present disclosure and which is herein incorporated by reference in its entirety.

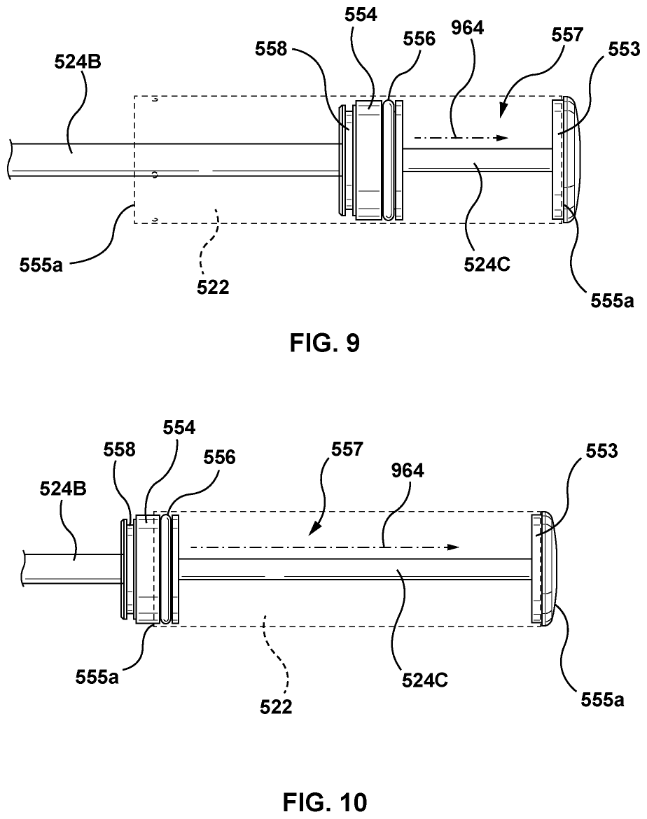

Turning now to FIGS. 7-10, the operation of the capsule 522 for radially compressing and deploying the capsule 522 will be described in more detail. FIG. 7 is a perspective view of the piston 554. FIGS. 8, 9, and 10 illustrate various positions of the capsule 552 relative to the piston 554 during deployment of the prosthetic heart valve 100. The prosthetic heart valve 100 is depicted in FIG. 8, but is not shown in FIGS. 9 and 10 for sake of clarity, as these figures are primarily provided to illustrate the relative movement of the capsule 552 relative to the piston 554. It will be understood by one of ordinary skill in the art that other delivery configurations of the prosthetic heart valve 100 are contemplated and the illustrated configuration is only exemplary.

The piston 554 is shown removed from the delivery system 520 in FIG. 7. The piston 554 is an annular component that defines an opening or central bore 533 such that the piston 554 is configured to be disposed over and attached to a distal end of the piston mount 524B. The piston 554 includes a first annular groove or recess 558 configured to receive the attachment tabs 112 of the prosthetic heart valve 100, as will be explained in more detail herein with respect to FIGS. 11-12A. The piston 554 also includes a second annular groove 560 on an outer surface thereof. A seal 556 (shown in FIGS. 8-10) is disposed within the second annular groove 560. The seal 556 is thus coupled to the piston 554 and functions to provide a fluid seal between the piston 554 and an inner surface of the capsule 522. The seal 556 may be, for example, an O-ring. When fluid is present in the annular chamber 557, the seal 556 prevents fluid from leaking out between the outer surface of the piston 554 and the inner surface of the capsule 522.

Referring now to FIG. 8, a side view of the distal portion of the delivery system 520 is shown. The distal portion includes the capsule 522 and the piston 554. In FIG. 8, the capsule 522 is in a first position relative to the piston 554 in which the piston 554 abuts against or is disposed directly adjacent to the capsule cap 553. In this first position, the capsule 522 is positioned so as to compressively retain at least a portion of the prosthetic heart valve 100. The prosthetic heart valve 100 is coupled to the piston 554 via the attachment tabs 112 being disposed within the first annular groove 558. In FIG. 8, the distal shaft 524C is concealed from view since the piston mount 524B extends thereover.

When the prosthetic heart valve 100 is loaded into the capsule 522 as shown in FIG. 8, at least a portion of the brim 115 of the prosthetic heart valve 100 may protrude from the capsule 522 prior to valve release. In an embodiment hereof, as depicted in FIG. 8, a suture 564 is disposed around the brim 115 to hold the brim 115 in a reduced diameter state for delivery. In this manner, the length of capsule 522 is minimized and the capsule 522 has a length less than the length of the prosthetic heart valve 100 in its reduced diameter state. In an embodiment, the suture 564 may be formed from a monofilament or plastic suture material, such as polypropylene. The suture 564 is a single, continuous elongated component that runs from the handle 527 of the inner steerable catheter 316 to the brim 115 of the prosthetic heart valve 100, around the brim 115 of the prosthetic heart valve 100, and back from the prosthetic heart valve 100 to the handle 527 of the inner steerable catheter 316 so that both ends of the suture 564 are accessible to the user. Stated another way, the suture 564 is a single, continuous elongated component that, when placed within the delivery system 520, integrally includes a first leg 564A, a second leg 564B, and a loop 564C formed therebetween the first and second legs 564A, 564B. The proximal ends of the first and second legs 564A, 564B extend proximally out of the handle 527 (as shown in FIG. 21) so as to be accessible to the user.

With further reference to the cross-sectional view of FIG. 5, the delivery system 520 also includes a dual lumen tube 565 for housing the suture 564. The dual lumen tube 565 extends within the annular lumen 532 defined between an outer surface of the innermost shaft assembly 524 and an inner surface of the inner steerable catheter 526. The first leg 564A of the suture 564 and the second leg 564B of the suture 564 extend through the dual lumen tube 565. The loop 564C of the suture 564 is disposed distally of the distal end of the dual lumen tube 565, and extends around the brim 115 of the prosthetic heart valve 100. The loop 564C of the suture 564 encircles or extends circumferentially around the brim 115 of the prosthetic heart valve 100 and is configured to hold the brim 115 in a reduced diameter state for delivery to the treatment site. The suture 564 is releasable to permit the brim 115 of the prosthetic heart valve 100 to return to an expanded or deployed state. More particularly, pulling on one or both ends of the suture 564 controls constriction/compression of the brim 115 of the prosthetic heart valve 100 and releasing/removing the suture 564 controls expansion/deployment of the brim 115 of the prosthetic heart valve 100. The operation and function of suture 564 will be described in more detail herein with reference to FIGS. 21-24.

With reference now to FIGS. 9 and 10, the capsule 522 is configured to be distally advanced relative to the piston 554 in order to incrementally release and deploy the frame 102 of the prosthetic heart valve 100 from the capsule 522. Via the manifold 525, fluid is injected through the innermost shaft assembly 524 in order to drive the capsule 522 distally. The prosthetic heart valve 100 may remain in a stationary longitudinal position relative to the native valve while the capsule 522 is driven distally, thereby increasing the precision of deployment. Hydraulic valve delivery systems consistent with embodiments hereof include, for example, those described in U.S. Pat. No. 9,034,032 to McLean et al., International Patent Application No. PCT/US21724/029549 to McLean et al., and U.S. Pat. No. 10,561,497 to Duffy et al., which are hereby incorporated by reference in their entirety.

More particularly, the manifold 525 may be connected to an external fluid source (not shown). The external fluid source is fluidly connected to the annular chamber 557 within the capsule 522 via the lumens of the flexible shaft 524A and the piston mount 524B (which are in fluid communication with each other). Fluid enters the annular chamber 557 via the outlet of the piston mount 524B, around the distal shaft 524C through the annular space or lumen 531 (see FIG. 5a) defined between the outer surface of the distal shaft 524C and the inner surface of the piston mount 524B. As shown by the directional arrow 964 in FIG. 9, as the annular chamber 557 fills with fluid, the capsule 522 is distally advanced with respect to the piston 554. FIG. 9 illustrates the capsule 522 at a second position relative to the piston 554, in which the piston 554 is disposed within the capsule 522 at approximately a midportion thereof. The annular chamber 557 between the piston 554 and the capsule cap 553 is filled with fluid from the external fluid source. The piston 554, which is attached and fixed to the piston mount 524B and the flexible shaft 524A, remains stationary as the capsule 522 and distal shaft 524C move in an axial direction. The piston 554 (and piston mount 524B and flexible shaft 524A) may be held in place by holding the manifold 525 stationary during fluid delivery.

The fluid continues to fill the annular chamber 557 until the capsule 522 reaches a third position relative to the piston 554 depicted in FIG. 10, in which the piston 554 is partially disposed within the capsule 522 and is directly adjacent to the proximal end 555b of the capsule 522. The annular chamber 557 between the piston 554 and the capsule cap 553 is filled with fluid from the external fluid source. At this third position, the first annular groove 558 of the piston 554 is no longer covered by the capsule 552. With the first annular groove 558 exposed, the attachment tabs 112 of the prosthetic heart valve 100 are permitted to decouple from the piston 554. Accordingly, via movement of the capsule 552, the prosthetic heart valve 100 is unsheathed from the capsule 522. When the capsule 522 no longer covers or extends over the attachment tabs 112, the attachment tabs 112 are free or permitted to pop out of the first annular groove 558 of the piston 554 to decouple the prosthetic heart valve 100 from the piston 554. Thus, once the prosthetic heart valve 100 is no longer covered by the capsule 522, the prosthetic heart valve 100 is permitted to radially self-expand towards the expanded configuration of FIG. 1.

FIG. 11 is a perspective view of the piston 554, with the piston 554 removed from the delivery system. Further, for sake of illustration only, FIG. 11 illustrates the attachment tabs 112 of the prosthetic heart valve 100 coupled to the piston 554. FIG. 12 is a side view of the outflow end 109 of the prosthetic heart valve 100 and the attachment tabs 112, and FIG. 12A is an enlarged view of an attachment tab 112.

Each attachment tab 112 has a L-shaped configuration. More particularly, each attachment tab 112 of the plurality of attachment tabs 112 includes a leg 1166, a base 1168, and a bend 1167 extending between the leg 1166 and the base 1168. The leg 1166 of each attachment tab 112 is attached to and extends from the second or outflow end 109 of the prosthetic heart valve 100. In the embodiment of FIGS. 11-12A, the leg 1166 extends parallel or generally parallel (i.e., within 5°) to the longitudinal axis L_{A} of the delivery system 520 and to the longitudinal axis of the prosthetic heart valve 100 mounted thereon. The base 1168 extends perpendicular or generally perpendicular (i.e., within 5°) to the longitudinal axis of the delivery system 520 and to the longitudinal axis of the prosthetic heart valve 100 mounted thereon. The bend 1167 of each attachment tab 112 forms an angle ⊖ between the leg 1166 and the base 1158, the angle being between 80 and 100 degrees. In an embodiment, the angle ⊖ is a right angle, or is 90°. In another embodiment, the angle ⊖ is generally 90° (i.e., within 5°). Each attachment tab 112 may be integrally formed with the frame 102 or may be separately formed from the frame 102 and subsequently attached thereto. Each attachment tab may be formed from a shape memory material, such as a nickel titanium alloy (e.g., Nitinol) and the L-shaped configuration may be formed during the shape setting operation of the frame 102.

The base 1168 of each attachment tab 112 is configured to extend into the annular groove 558 of the piston 554. The annular groove 558 has a sufficient width W and a sufficient depth D to receive the base 1168 of each attachment tab 112. Stated another way, the annular groove 558 is configured or sized such that it is deep and wide enough to accommodate the dimensions of the base 1168 of each attachment tab 112. In an embodiment the width W is between 0.010 inches to 0.025 inches, while a width W₁ of the base 1168 of each attachment tab 112 is between 0.005 inches to about 0.020 inches. In an embodiment the depth D of the annular groove is between 0.030 inches to 0.060 inches, while a depth D₁ of the base 1168 of each attachment tab 112 is between 0.025 inches to about 0.050 inches. The annular groove 558 is thus sized or configured to accommodate or receive the base 1168 such that the base 1168 of each attachment tab 112 may drop or extend into the annular groove 558 to thereby couple the prosthetic heart valve 100 to the piston 554. As best shown on FIG. 11, when the base 1168 of each attachment tab 112 extends into the annular groove 558, an inner surface 1163A (see FIG. 12) of the base 1168 abuts against and contacts an interior wall surface 1163B (see FIG. 11) of the annular groove 558, such that the bend 1167 of the attachment tab 112 hooks or clips onto the annular groove 558. In a delivery configuration of the delivery system 520, the plurality of attachment tabs 112 are disposed within the annular groove 558 of the piston 554 and the capsule 522 covers and constrains the prosthetic heart valve 100 in a radially collapsed configuration, with the capsule 522 extending over the annular groove 558 of the piston 554 and the attachment tabs 112 received therein.

Further, as best shown in the side view of the piston 554 in FIG. 11A, the piston 554 includes a reduced outer diameter portion proximal to the annular groove 558 such that the attachment tabs 112 do not increase the outer profile of the piston 554 when the attachment tabs 112 are coupled to the piston 554. More particularly, the piston 554 includes a first longitudinal portion 580 between the annular groove 558 and a proximal end 581 thereof and includes a second longitudinal portion 582 distal to the annular groove 558. The first longitudinal portion 580 has a first outer diameter OD₁ which is less than a second outer diameter OD₂ of the second longitudinal portion 582. The first outer diameter OD₁ is sized smaller than the second outer diameter OD₂ so that the overall profile of the piston 554 does not increase when the attachment tabs 112 extend over the first longitudinal portion 580 when coupled to the piston 554. That is, the sum of the first outer diameter OD₁ and twice a depth or thickness Ti of a leg 1166 of an attachment tab 112 is approximately equal to the second outer diameter OD₂.

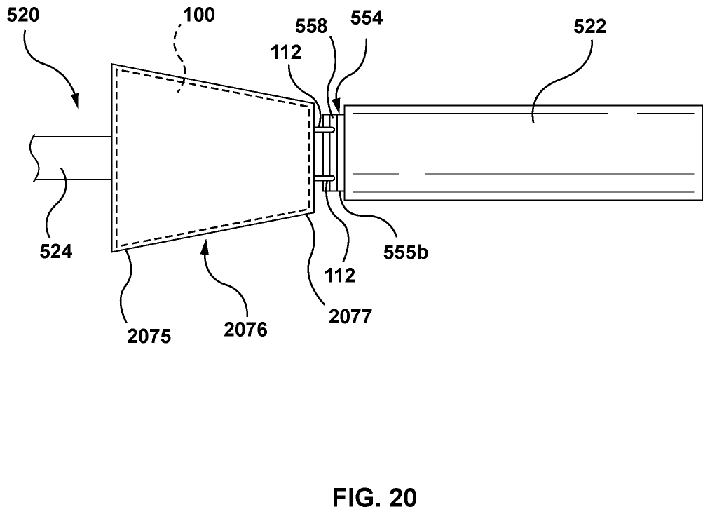

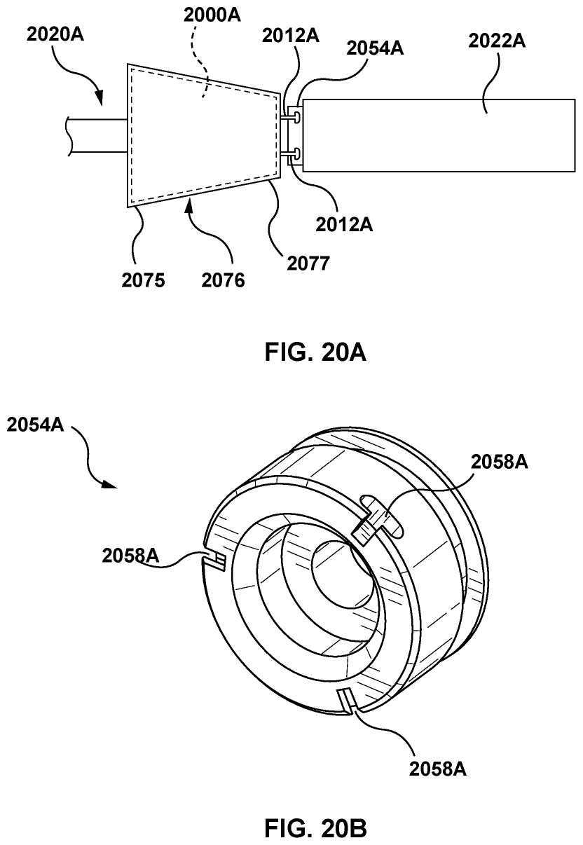

Since the annular groove 558 is continuous and extends around the entire circumference of the piston 554, the method of coupling the prosthetic heart valve 100 to the piston 554 is greatly simplified. Rather than including a dedicated recess or slot for each attachment tab 112, the annular groove 558 receives all of the attachment tabs 112. More particularly, it is difficult for an operator to load attachment structures into a plurality of dedicated recesses or slots during loading because such loading requires precise circumferentially alignment between the attachment structures and the plurality of dedicated recesses or slots. An operator must rotate the delivery system and the piston thereof in order to precisely align the plurality of dedicated recesses or slots with the attachment structures of the prosthetic heart valve, and visualization of the positioning of the plurality of dedicated recesses or slots may be obstructed or hindered. With the annular groove 558, however, the process of positioning or placing the attachment tabs 112 into the annular groove 558 is simplified because each attachment tab 112 may be disposed into the annular groove 558 at any relative circumferential position between the prosthetic heart valve 100 and the piston 554. This simpler method of attachment between the prosthetic heart valve 100 and the delivery system 520 makes it easier for the operator to consistently achieve correct loading of the prosthetic heart valve 100 onto the delivery system 520. The improved loading of the prosthetic heart valve 100 is further illustrated in FIG. 20, described below, as compared to FIGS. 20A and 20B which illustrate loading difficulties with T-shaped attachment tabs rather than the attachment tabs 112 of the present invention.

Another benefit of the continuous, annular groove 558 is that it allows for more consistent release of the prosthetic heart valve 100 upon full deployment from the piston 554. If each attachment structure is each disposed within a dedicated recess or slot, the attachment structure may become stuck within the dedicated recess or slot during deployment. The continuous, annular groove 558 permits a smoother and more consistent release of the attachment tabs 112 from the piston 554 when the capsule 522 no longer covers and constrains the annular groove 558.

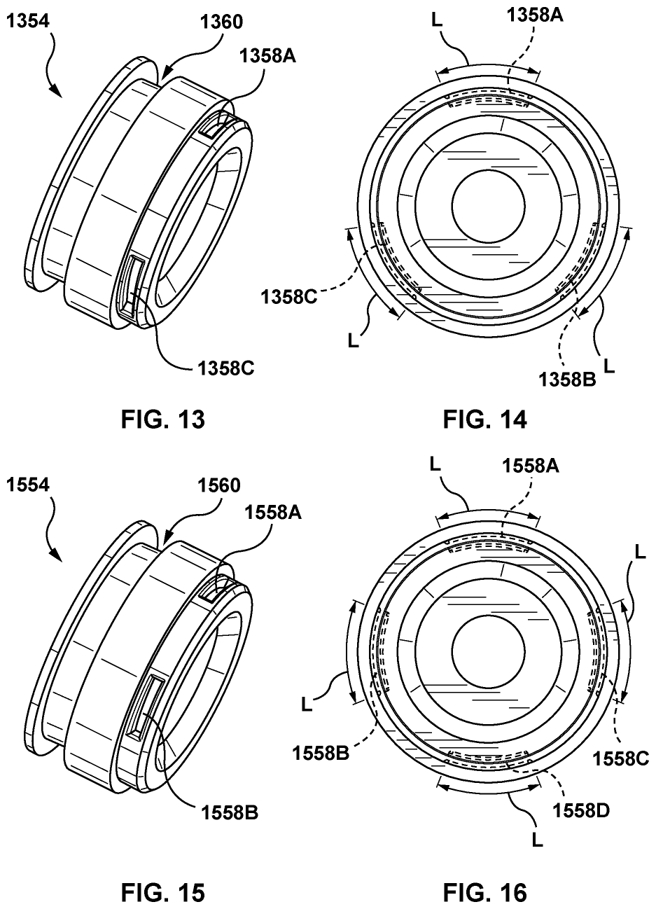

Although the continuity of the annular groove 558 is generally believed to simplify the loading process to the greatest extent possible, in another embodiment hereof, the piston may include a plurality of circumferentially-extending grooves for receiving the attachment tabs 112 of the prosthetic heart valve 100. More particularly, another embodiment hereof is depicted in FIGS. 13 and 14. FIG. 13 is a perspective view of a piston 1354 that may be utilized in the delivery system 520 described above rather than the piston 554. Rather than the continuous, annular groove 558 of the piston 554, the piston 1354 include a plurality of discontinuous, circumferentially-extending grooves 1358A, 1358B, 1358C on an outer surface thereof for receiving the attachment tabs 112 of the prosthetic heart valve 100. FIG. 14 is an end view of the piston 1354.

In the delivery configuration of the delivery system 520, the base 1168 of each attachment tab 112 extends into a corresponding circumferentially-extending groove of the plurality of circumferentially-extending grooves 1358A, 1358B, 1358C of the piston 1354 and the capsule 522 covers and constrains the prosthetic heart valve 100 in a radially collapsed configuration, with the capsule 522 extending over the plurality of circumferentially-extending grooves 1358A, 1358B, 1358C and the attachment tabs 112 received therein. The base 1168 of each attachment tab 112 has a first length Lᵢ. In this context, the length of each attachment tab 112 is an amount or distance that each attachment tab 112 extends in a circumferential direction. In an embodiment, each circumferentially-extending groove of the plurality of circumferentially-extending grooves 1358A, 1358B, 1358C has a length L that is at least 300% greater than the first length Lᵢ of the base 1168 of an attachment tab of the plurality of attachment tabs 112. For example, in an embodiment, each circumferentially-extending groove of the plurality of circumferentially-extending grooves 1358A, 1358B, 1358C has a length L of approximately 0.080 inches and the first length Lᵢ of the base 1168 of an attachment tab of the plurality of attachment tabs 112 is approximately 0.020 inches. In another embodiment, each circumferentially-extending groove of the plurality of circumferentially-extending grooves 1358A, 1358B, 1358C has a length L that is between 300% and 500% greater than the first length Lᵢ of the base 1168 of an attachment tab of the plurality of attachment tabs 112.

Although the piston 1354 includes dedicated circumferentially-extending grooves 1358A, 1358B, 1358C for receiving a corresponding attachment tab, the method of coupling the prosthetic heart valve 100 to the piston 1354 is simplified relative to prior art systems due to the size of the circumferentially-extending grooves 1358A, 1358B, 1358C and the configuration of the attachment tabs 112. Since each circumferentially-extending groove 1358A, 1358B, 1358C is at least 300% longer than the first length Lᵢ of the base 1168 of an attachment tab 112, the process of positioning or placing the attachment tabs 112 into the circumferentially-extending grooves 1358A, 1358B, 1358C is simplified because the longer lengths of the circumferentially-extending grooves 1358A, 1358B, 1358C do not require precise circumferentially alignment between the attachment tabs 112 and the plurality of circumferentially-extending grooves 1358A, 1358B, 1358C. Another benefit of the size of the circumferentially-extending grooves 1358A, 1358B, 1358C is that the longer grooves allow for more consistent release of the prosthetic heart valve 100 upon full deployment from the piston 554. Since each circumferentially-extending groove 1358A, 1358B, 1358C is at least 300% longer than the first length Lᵢ of the base 1168 of an attachment tab 112, each attachment tabs 112 is not likely to be stuck within its respective circumferentially-extending grooves 1358A, 1358B, 1358C.

In an embodiment, in which the prosthetic heart valve 100 includes a total of three attachment tabs 112, the piston 1354 includes a total of three circumferentially-extending grooves 1358A, 1358B, 1358C. Stated another way, the number of circumferentially-extending grooves is equal to the number of the attachment tabs 112. The circumferentially-extending grooves 1358A, 1358B, 1358C are disposed at approximately equal intervals from each other around a perimeter of the piston 1354 and thus are located 1200 or approximately 1200 (i.e., within 5°) apart from each other around the circumference of the piston 1354. However, it is not required that the prosthetic heart valve 100 include three attachment tabs 112. In another embodiment, the prosthetic heart valve 100 may include four attachment tabs and the piston may include a total of four circumferentially-extending grooves. More particularly, another embodiment hereof is depicted in FIGS. 15 and 16. FIG. 15 is a perspective view of a piston 1554 that may be utilized in the delivery system 520 described above rather than the piston 554. Rather than the continuous, annular groove 558 of the piston 554, the piston 1554 include a plurality of discontinuous, circumferentially-extending grooves 1558A, 1558B, 1558C, 1558D on an outer surface thereof for receiving four attachment tabs 112 of the prosthetic heart valve 100. FIG. 16 is an end view of the piston 1554. The circumferentially-extending grooves 1558A, 1558B, 1558C, 1558D are disposed at approximately equal intervals from each other around a perimeter of the piston 1554 and thus are located 90° or approximately 90° (i.e., within 5°) apart from each other around the circumference of the piston 1554.

In the delivery configuration of the delivery system 520, the base 1168 of each attachment tab 112 extends into a corresponding circumferentially-extending groove of the plurality of circumferentially-extending grooves 1558A, 1558B, 1558C, 1558D of the piston 1554 and the capsule 522 covers and constrains the prosthetic heart valve 100 in a radially collapsed configuration, with the capsule 522 extending over the plurality of circumferentially-extending grooves 1558A, 1558B, 1558C, 1558D and the attachment tabs 112 received therein. The base 1168 of each attachment tab 112 has the first length Lᵢ. In this context, the length of each attachment tab 112 is measured in amount that each attachment tab 112 extends in a circumferential direction. Each circumferentially-extending groove of the plurality of circumferentially-extending grooves 1558A, 1558B, 1558C, 1558D has a length L that is at least 300% greater than the first length Lᵢ of the base 1168 of an attachment tab of the plurality of attachment tabs 112. In another embodiment, each circumferentially-extending groove of the plurality of circumferentially-extending grooves 1558A, 1558B, 1558C, 1558D has a length L that is between 300% and 500% greater than the first length Lᵢ of the base 1168 of an attachment tab of the plurality of attachment tabs 112.

Although the piston 1554 includes dedicated circumferentially-extending grooves 1558A, 1558B, 1558C, 1558D for receiving a corresponding attachment tab, the method of coupling the prosthetic heart valve 100 to the piston 1554 is simplified relative to prior art systems due to the size of the circumferentially-extending grooves 1558A, 1558B, 1558C, 1558D and the configuration of the attachment tabs 112. Since each circumferentially-extending groove 1558A, 1558B, 1558C, 1558D is at least 300% longer than the first length Lᵢ of the base 1168 of an attachment tab 112, the process of positioning or placing the attachment tabs 112 into the circumferentially-extending grooves 1558A, 1558B, 1558C, 1558D is simplified because the longer lengths of the circumferentially-extending grooves 1558A, 1558B, 1558C, 1558D do not require precise circumferentially alignment between the attachment tabs 112 and the plurality of circumferentially-extending grooves 1558A, 1558B, 1558C, 1558D. Another benefit of the size of the circumferentially-extending grooves 1558A, 1558B, 1558C, 1558D is that the longer grooves allow for more consistent release of the prosthetic heart valve 100 upon full deployment from the piston 554. Since each circumferentially-extending groove 1558A, 1558B, 1558C, 1558D is at least 300% longer than the first length Lᵢ of the base 1168 of an attachment tab 112, each attachment tabs 112 is not likely to be stuck within its respective circumferentially-extending grooves 1558A, 1558B, 1558C, 1558D.

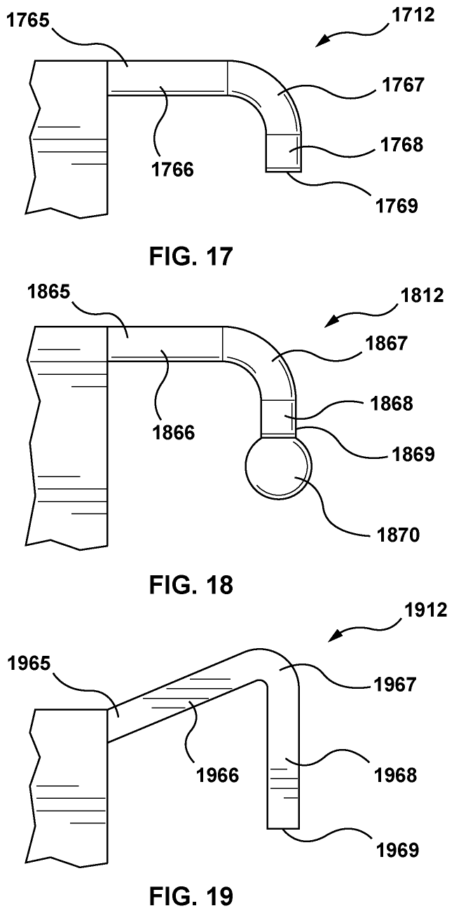

The configuration of the attachment tabs 112 may also vary from the specific configuration depicted in FIGS. 11-12A. More particularly, an attachment tab according to another embodiment hereof is depicted in FIG. 17. FIG. 17 is an enlarged view of an attachment tab 1712. Each attachment tab 1712 is configured for coupling to the annular groove 558 of the piston 554, one of the circumferentially-extending grooves 1358A, 1358B, 1358C of the piston 1354, or one of the circumferentially-extending grooves 1558A, 1558B, 1558C, 1558D of the piston 1554.

The attachment tab 1712 has a modified L-shaped configuration. More particularly, the attachment tab 1712 includes a first end 1765 which is attached to and extends from the prosthetic heart valve and a second or free end 1769. The attachment tab 1712 includes a leg 1766, a base 1768, and a bend 1767 extending between the leg 1766 and the base 1768. In the embodiment of FIG. 17, the leg 1766 extends parallel or generally parallel (i.e., within 5°) to the longitudinal axis L_{A} of the delivery system 520 and to the longitudinal axis of the prosthetic heart valve mounted thereon. The base 1768 extends perpendicular or generally perpendicular (i.e., within 5°) to the longitudinal axis of the delivery system 520 and to the longitudinal axis of the prosthetic heart valve mounted thereon. The bend 1767 of each attachment tab 1712 has a curved profile. The curved profile of the bend 1767 is rounder or smoother than the profile of the bend 1167 of the attachment tab 112 and may reduce potential for the attachment tab 1712 to fracture during the loading process. An angle ⊖ is formed between the leg 1766 and the base 1768, the angle being between 80 and 100 degrees. In an embodiment, the angle ⊖ is a right angle, or is 90°. In another embodiment, the angle ⊖ is generally 90° (i.e., within 5°).

An attachment tab according to another embodiment hereof is depicted in FIG. 18. FIG. 18 is an enlarged view of an attachment tab 1812. The attachment tab 1812 is the same as the attachment tab 1712 except that the attachment tab 1812 includes a spherical ball 1870. Each attachment tab 1812 is configured for coupling to the annular groove 558 of the piston 554, one of the circumferentially-extending grooves 1358A, 1358B, 1358C of the piston 1354, or one of the circumferentially-extending grooves 1558A, 1558B, 1558C, 1558D of the piston 1554.

More particularly, the attachment tab 1812 has a modified L-shaped configuration. More particularly, the attachment tab 1812 includes a first end 1865 which is attached to and extends from the prosthetic heart valve and a second or free end 1869. The attachment tab 1812 includes a leg 1866, a base 1868, and a bend 1867 extending between the leg 1866 and the base 1868. In the embodiment of FIG. 18, the leg 1866 extends parallel or generally parallel (i.e., within 5°) to the longitudinal axis L_{A} of the delivery system 520 and to the longitudinal axis of the prosthetic heart valve mounted thereon. The base 1868 extends perpendicular or generally perpendicular (i.e., within 5°) to the longitudinal axis of the delivery system 520 and to the longitudinal axis of the prosthetic heart valve mounted thereon. The bend 1867 of each attachment tab 182 has a curved profile. The curved profile of the bend 1867 is rounder or smoother than the profile of the bend 1167 of the attachment tab 112 and may reduce potential for the attachment tab 1812 to fracture during the loading process. An angle ⊖ is formed between the leg 1866 and the base 1868, the angle being between 80 and 100 degrees. In an embodiment, the angle ⊖ is a right angle, or is 90°. In another embodiment, the angle ⊖ is generally 90° (i.e., within 5°).

The second or free end 1869 of the attachment tab 1812 includes the spherical ball 1870. The spherical outer surface of the spherical ball 1870 reduces friction between the attachment tab 1812 and the annular or circumferentially-extending groove of the piston, thus facilitating easier or more reliable release of the attachment tab 1812 from the annular or circumferentially-extending groove of the piston as the attachment tab 1812 is released from the piston during valve deployment. The spherical ball 1870 may be attached to the base 1868 via any suitable mechanical process, including but not limited to via a weld, adhesive, or other bonding mechanism. In an embodiment, the spherical ball 1870 may be formed integrally with the base 1868 by heating the base 1868 with a laser to melt or form the end of the base into a spherical geometry.

An attachment tab according to another embodiment hereof is depicted in FIG. 19. FIG. 19 is an enlarged view of an attachment tab 1912. Each attachment tab 1912 is configured for coupling to the annular groove 558 of the piston 554, one of the circumferentially-extending grooves 1358A, 1358B, 1358C of the piston 1354, or one of the circumferentially-extending grooves 1558A, 1558B, 1558C, 1558D of the piston 1554.

The attachment tab 1912 has a modified L-shaped configuration. More particularly, the attachment tab 1912 includes a first end 1965 which is attached to and extends from the prosthetic heart valve and a second or free end 1969. The attachment tab 1912 includes a leg 1966, a base 1968, and a bend 1967 extending between the leg 1966 and the base 1968. In the embodiment of FIG. 19, the leg 1966 extends at an acute angle 62 relative to the longitudinal axis L_{A} of the delivery system 520 and to the longitudinal axis of the prosthetic heart valve mounted thereon. In an embodiment, the acute angle 62 is between 10° and 30°. The base 1968 extends perpendicular or generally perpendicular (i.e., within 5°) to the longitudinal axis of the delivery system 520 and to the longitudinal axis of the prosthetic heart valve mounted thereon. An angle ⊖ is formed between the leg 1966 and the base 1968, the angle being between 50 and 80 degrees. The angled configuration of the leg 1966 may improve the position-ability of the attachment tab 1912 into the annular or circumferentially-extending groove of the piston, and may faciliate easier or more reliable release of the attachment tab 1912 from the annular or circumferentially-extending groove of the piston as the attachment tab 1912 is released from the piston during valve deployment.

FIG. 20 is a schematic side view representing a method of loading the prosthetic heart valve 100 into the capsule 522 of the delivery system 520 and thereby releasably coupling the prosthetic heart valve 100 to the delivery system 520 for delivery thereof. The prosthetic heart valve 100 is first crimped down to a reduced diameter by advancing the prosthetic heart valve 100 into a loading cone or funnel 2076. In another embodiment (not shown), the prosthetic heart valve 100 may be crimped down to a reduced diameter via a radial crimper. The loading funnel 2076 is a conical or tapered component that includes a first end 2075 having a first diameter and a second end 2077 having a second diameter that is less than the first diameter. The prosthetic heart valve 100 in its expanded or shape set configuration is positioned into the first end 2075 of the loading funnel 2076 and advanced through the loading funnel 2076 towards the second end 2077 thereof to reduce its diameter. With the prosthetic heart valve 100 disposed therein, the assembly of the loading funnel 2076 and the prosthetic heart valve 100 disposed therein is placed over the innermost shaft assembly 524 of the delivery system 520 in a chilled saline bath to soften the frame 102 prior to advancement into the loading funnel 2076. The assembly of the loading funnel 2076 and the prosthetic heart valve 100 is positioned proximal to a proximal end of the capsule 522. The prosthetic heart valve 100 is advanced through the loading funnel 2076 until attachment tabs 112 of the prosthetic heart valve 100 extend or protrude from the second end 2077 of the loading funnel 2076.

At this stage or point of the method of loading, the delivery system 520 is still located within the saline bath, and the assembly of the loading funnel 2076 and the prosthetic heart valve 100 is positioned proximal to the open or proximal end 555b of the capsule 522. The operator then positions the attachment tabs 112 of the heart valve prosthesis 100 into the annular groove 558 of the piston 554 of the delivery system 520. Once the attachment tabs 112 are disposed into the annular groove 558, the base 1168 of each attachment tab 112 extends into the annular groove 558 of the piston 554 and the leg 1166 of each attachment tab 112 extends generally parallel to the longitudinal axis of the delivery system 520 and to the longitudinal axis of the heart valve prosthesis 100. Since the annular groove 558 is continuous and extends around the entire perimeter of the piston 554, each attachment tab 112 may be disposed into the annular groove 558 at any relative circumferential position between the prosthetic heart valve 100 and the piston 554. Precise circumferential alignment between the attachment tabs 112 and the annular groove 554 is not required, which makes it easier for the operator to consistently achieve correct loading of the prosthetic heart valve 100 onto the piston 554 of the delivery system 520.

After the attachment tabs 112 of the heart valve prosthesis 100 are positioned into the annular groove 558 of the piston 554, the prosthetic heart valve 100 is then pulled into the capsule 522 via distal movement of the piston 554. Stated another way, distal movement of the piston 554 pulls or retracts the prosthetic heart valve 100 into the capsule 522. Once the prosthetic heart valve 100 is loaded into the capsule 522, the delivery system 520 is in the delivery configuration and the frame 102 of the prosthetic heart valve 100 is positioned into the capsule such that the capsule 522 covers and constrains the prosthetic heart valve 100 in a radially collapsed configuration, with the capsule 522 extending over the annular groove 558 of the piston 554 and the attachment tabs 112 received therein.

The improved method of loading of the prosthetic heart valve 100 is apparent when comparing FIG. 20 with FIGS. 20A and 20B, which illustrate the loading difficulties with T-shaped attachment tabs known in the art rather than the attachment tabs 112 of the present invention. More particularly, FIG. 20A is a schematic side view representing a method of loading a prosthetic heart valve 2000A having T-shaped attachment tabs 2012A into a capsule 2022A of delivery system 2020A and thereby releasably coupling the prosthetic heart valve 2000A to the delivery system 2020A for delivery thereof. The prosthetic heart valve 2000A is first crimped down to a reduced diameter by advancing the prosthetic heart valve 2000A into the loading cone or funnel 2076. The prosthetic heart valve 2000A is advanced through the loading funnel 2076 until the T-shaped attachment tabs 2012A of the prosthetic heart valve 2000A extend or protrude from the second end 2077 of the loading funnel 2076. The operator then positions the T-shaped attachment tabs 2012A of the heart valve prosthesis 2000A into the dedicated T-shaped recesses 2058A of the piston 2054A, which are best illustrated in the perspective view of the piston 2054A in FIG. 20B. Precise circumferential alignment between the attachment tabs 2012A and the dedicated T-shaped recesses 2058A is required, which makes it difficult for the operator to consistently achieve correct loading of the prosthetic heart valve 100 onto the piston 554 of the delivery system 520. However, as described above with respect to FIG. 20, the piston 554 and attachment tabs 112 of the present invention simplify the loading process for the operator because the annular groove 558 is continuous and extends around the entire circumference of the piston 554. Rather than including a dedicated recess for each attachment tab 112, the annular groove 558 receives all of the attachment tabs 112 and each attachment tab 112 may be disposed into the annular groove 558 at any relative circumferential position between the prosthetic heart valve 100 and the piston 554.

In various methods of the present disclosure, a system is prepared by crimping and loading the prosthetic heart valve 100 onto the piston 554 of the delivery system 520, or alternatively the piston 1354 or the piston 1554. In delivery configuration, the system is directed to a target site via transcatheter procedure. In an example, the target site is a native heart valve or previous implanted prosthesis heart valve. In the illustrated example, the target site may be a mitral valve via the left atrium. FIGS. 21-24 illustrate an exemplary method of delivering the prosthetic heart valve 100 to a native mitral valve annulus with the delivery system 520.

As shown in FIG. 21, the delivery system 520 may be delivered to the target site via an introducer sheath 2172 having a hemostasis valve on a proximal end thereof. In an embodiment, the target site is a native mitral valve and the introducer sheath 2172 is tracked to the right atrium via the inferior vena cava. The introducer sheath 2172 may be used to make a transeptal entry into the left atrium across the septum. The introducer sheath 2172 may be subsequently withdrawn after the delivery system 310 is positioned across the septum. The introducer sheath 2172 may be steerable or pre-shaped in a configuration suitable for the particular approach to the target valve. The proximal ends of the first and second legs 564A, 564B of the suture 564 extend proximally out of the handle 527, as shown in FIG. 21, so as to be accessible to the user.

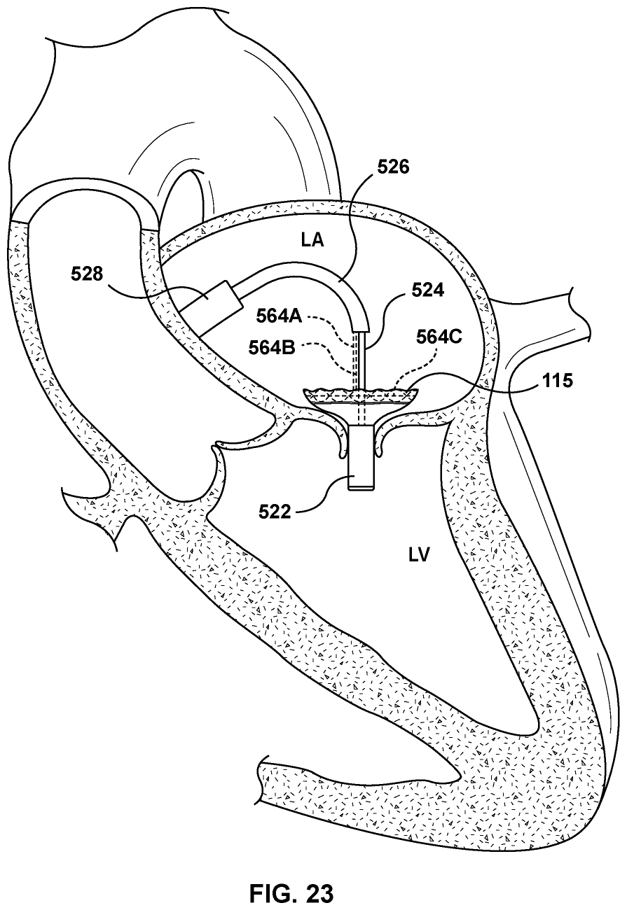

FIGS. 22-24 are sectional cut-away views of a heart illustrating a transseptal approach for delivering and positioning the prosthetic heart valve 100 using the delivery system 520 and in accordance with an embodiment hereof. It is not necessary that the following operations of method of use occur in the order in which they are described. With reference to FIG. 22, the delivery system 520 is shown after having been introduced into the vasculature via a percutaneous entry point, e.g., the Seldinger technique, and having been tracked through the vasculature and into the left atrium so that capsule 522 is positioned proximate the native mitral valve MV. Intravascular access to the right atrium RA may be achieved via a percutaneous access site to femoral venous access up to the inferior vena cava, or other known access routes. Thereafter, a guidewire (not shown) is advanced through the circulatory system, eventually arriving at the heart. The guidewire is directed into the right atrium, traverses the right atrium and is made to puncture with the aid of a transeptal needle or pre-existing hole, the atrial septum, thereby entering the left atrium LA. Once the guidewire is positioned, the endoluminal entry port and the atrial septum are dilated to permit entry of an introducer sheath (not shown) into the left atrium LA. Thereafter, the introducer sheath 2172 is advanced over the guidewire and into the left atrium LA through the punctured atrial septum and positioned proximate or upstream to the native mitral valve MV. The guidewire is removed and the delivery system 520 is advanced through the introducer sheath 2172. Although described as a transfemoral antegrade approach for percutaneously accessing the mitral valve, the prosthetic heart valve 100 may be positioned within the desired area of the heart via entry other different methods such as a transseptal antegrade approach via a thoracotomy for accessing the mitral valve. In addition, although described with the use of a guidewire, in another embodiment hereof the introducer sheath 2172 may access the right atrium without the use of a guidewire.

In FIG. 22, the distal portion of delivery system 520 is shown positioned within the native mitral valve MV with the capsule 522 and the loop 564C of the suture 564 concurrently holding the prosthetic heart valve 100 in a reduced diameter state. In embodiments, the introducer sheath 2172 may be retracted across the septum after the capsule 522, the inner steerable catheter 526, and the outer steerable catheter 528 have crossed the septum. Thus, the introducer sheath 2172 is not shown in FIGS. 22-24. By manipulating the inner and outer distal flex components 540, 550, respectively, via the respective handles of the inner and outer steerable catheters 526, 528, outside the vasculature, a clinician may remotely manipulate and steer the distal portion of the delivery system 520 within the confined space of the left atrium LA.

Once the prosthetic heart valve 100 is positioned within the native mitral valve MV, tension on the suture 564 is released and the brim 115 of the prosthetic heart valve 100 is no longer constrained in the reduced diameter state by the loop 564C of the suture 564 as shown in FIG. 23. Slack of the suture 564 permits the brim 115 of the prosthetic heart valve 100 to return to an expanded state within an atrial area of the native mitral valve MV. Actuation of the loop 564C of the suture 564 and subsequent deployment of the brim 115 of the prosthetic heart valve 100 may be considered a first stage of deployment of a two-stage deployment process for the prosthetic heart valve 100. After the brim 115 is radially expanded, the capsule 522 maintains the frame 102 of the prosthetic heart valve 100 in the reduced diameter state. Although slackened, the loop 564C of the suture 564 notably still remains around the brim 115. Therefore, the brim 115 may be returned to its reduced diameter state by applying tension to the suture 564 and the brim 115 may be repositioned as needed.

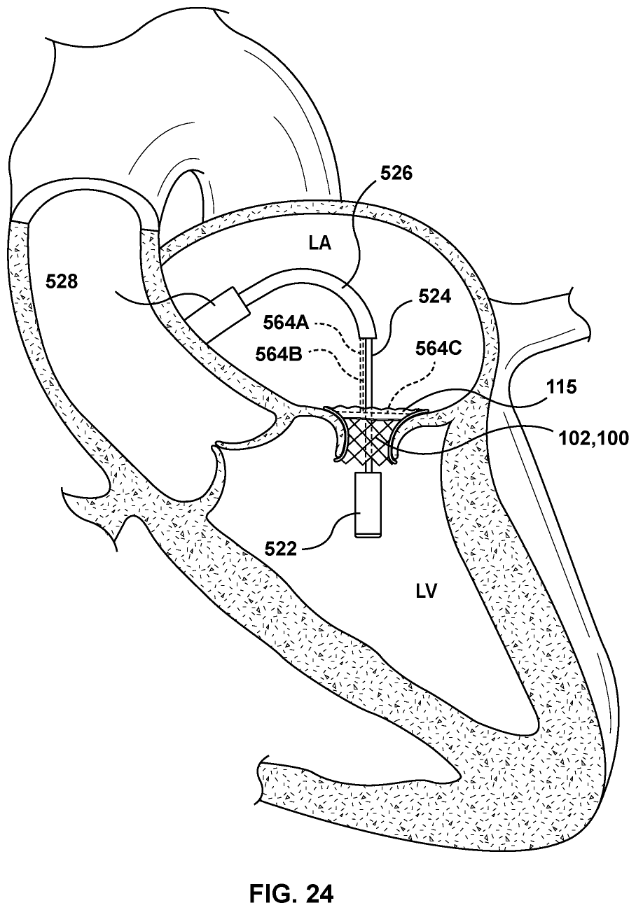

FIG. 24 is an illustration of a second stage of deployment of the prosthetic heart valve 100 in which the capsule 522 has been distally advanced to deploy the frame 102 of the prosthetic heart valve 100 as described above with respect to FIGS. 8-10. As described above, hydraulics are used to deploy the capsule 522 distally and away from the prosthetic heart valve 100. More particularly, fluid is injected through the innermost shaft assembly 524 in order to drive the capsule 522 distally as described above. The capsule 522 is distally advanced to expose and release the frame 102 of the prosthetic heart valve 100, thereby permitting the frame 102 of the prosthetic heart valve 100 to return to an expanded state. Distal advancement of the capsule 522 eventually uncovers or unsheathes the prosthetic heart valve 100, such that the prosthetic heart valve 100 decouples from the piston 554 and radially expands towards the expanded configuration of FIG. 1. As described above, pistons 554, 1354, 1554 described herein are configured to provide smoother and more consistent release of the attachment tabs 112 of the prosthetic heart valve 100 from the piston 554 due to the continuous, annular groove 558 thereof (or the circumferentially-extending grooves of pistons 1354, 1554) when the capsule 522 no longer covers and constrains the annular groove 558.

If the valve deployment is successful, the suture 564 is removed by pulling on one end of the suture 564 (either the end associated with the first leg 564A or the end associated with second leg 564B) until the entire suture 564 is pulled through the dual lumen tube 565 and removed from the delivery system. Once the prosthetic heart valve 100 is fully deployed and released from the delivery system 520, the capsule 522 can be proximally withdrawn through the prosthetic heart valve 100 and withdrawn from the patient in the same manner that the delivery system 520 was delivered.

During valve deployment described above, it may become necessary to reposition, recover, recapture, or retrieve a partially deployed prosthetic heart valve 100. Partially deployed, as used herein, refers to a delivery state of the prosthetic heart valve 100 in which the capsule 522 has been distally advanced such that at least a portion of the frame 102 has been deployed, while the attachment tabs 112 are still coupled to the piston 554 with the capsule 522 disposed thereover. Bailout procedures may become necessary after failed valve deployment when the prosthetic heart valve 100 is mislocated or damaged during deployment. As described above, the capsule 522 may be driven proximally via the tension cable 530 and the recapture piston 590 in order to recapture the frame 102. Stated another way, after a failed valve deployment, the hydraulic system of the delivery system 520 may be used to draw the prosthetic heart valve 100 back into the capsule 522 as far as possible. The brim 115 will protrude proximally from the capsule 522. To ensure that the brim 115 can be drawn back across the septum without damage to the patient anatomy, the brim 115 is recaptured and returned to its reduced diameter state by applying tension to the suture 564. After the brim 115 has been recaptured by the suture 564 and the frame 102 has been recaptured by the capsule 522, the outer steerable catheter 528, the inner steerable catheter 526, and the innermost shaft assembly 524 are drawn back together into the introducer sheath 2172. This may be accomplished by relative movement between the outer steerable catheter 528, the inner steerable catheter 526, and the innermost shaft assembly 524 against the introducer sheath 2172. For example, the outer steerable catheter 528, the inner steerable catheter 526, and the innermost shaft assembly 524 may be advanced while the introducer sheath 2172 is maintained in a stationary position. Alternatively, the introducer sheath 2172 may be advanced while maintaining the outer steerable catheter 528, the inner steerable catheter 526, and the innermost shaft assembly 524 in a stationary position. In another example, the outer steerable catheter 528, the inner steerable catheter 526, and the innermost shaft assembly 524 may be retracted while the introducer sheath 2172 is advanced. The introducer sheath 2172 may pull the prosthetic heart valve 100 back across the septum of the patient for withdrawal without injury to patient anatomy, and the introducer sheath 2172 and the delivery system 520 can be removed from the patient.

Although embodiments hereof are described with exemplary prosthetic heart valves and an exemplary delivery system, aspects of the present disclosure are not intended to be limited to the examples described herein. For example, although described herein with respect to a prosthetic heart valve having inner and outer frames, the present disclosure may be applied to any prosthetic heart valve which is coupled to a piston of a delivery system via a plurality of attachment structures. In addition, although described herein with respect to a delivery system having inner and outer steerable catheters, and other features, the present disclosure may be applied to any delivery system for coupling a prosthetic heart valve thereto. As another example, aspects of the present disclosure are not intended to be limited to delivery systems having capsules that are hydraulically controlled.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A system comprising:
a valve prosthesis including a frame and a prosthetic valve disposed within the frame, the frame being self-expanding and including a plurality of attachment tabs extending from a first end of the frame; and
a delivery system for percutaneously delivering the valve prosthesis, the delivery system including a shaft, a piston disposed over the shaft, and a capsule, the capsule being movable relative to the piston, wherein the piston includes an annular groove on an outer surface thereof,
wherein in a delivery configuration of the delivery system the plurality of attachment tabs are disposed within the annular groove of the piston and the capsule covers and constrains the valve prosthesis in a radially collapsed configuration, with the capsule extending over the annular groove of the piston and the attachment tabs received therein.

2. The system of claim 1, wherein each attachment tab of the plurality of attachment tabs has an L-shaped configuration and includes a leg, a base, and a bend extending between the leg and the base, the base extending generally perpendicular to a longitudinal axis of the delivery system and being configured to extend into the annular groove of the piston.

3. The system of claim 2, wherein the leg of each attachment tab of the plurality of attachment tabs extends generally parallel to the longitudinal axis of the delivery system.

4. The system of claim 2, wherein the leg of each attachment tab of the plurality of attachment tabs extends at an acute angle relative to the longitudinal axis of the delivery system.

5. The system of claim 2, wherein the bend of each attachment tab of the plurality of attachment tabs has a curved profile.

6. The system of claim 2, wherein the bend of each attachment tab of the plurality of attachment tabs forms an angle between the leg and the base, the angle being between 80 and 100 degrees.

7. The system of claim 2, wherein each attachment tab of the plurality of attachment tabs includes a spherical ball disposed at a free end thereof, the spherical ball being attached to the base.

8. A system comprising:
a valve prosthesis including a frame and a prosthetic valve disposed within the frame, the frame being self-expanding and including a plurality of attachment tabs extending from a first end of the frame; each attachment tab of the plurality of attachment tabs having an L-shaped configuration and including a leg, a base, and a bend extending between the leg and the base, the base extending generally perpendicular to a longitudinal axis of the delivery system and the base having a first length; and
a delivery system for percutaneously delivering the valve prosthesis, the delivery system including a shaft, a piston disposed over the shaft, and a capsule, the capsule being movable relative to the piston, wherein the piston includes a plurality of circumferentially-extending grooves on an outer surface thereof, each circumferentially-extending groove having a length that is at least 300% greater than the first length of the base of an attachment tab of the plurality of attachment tabs,
wherein in a delivery configuration of the delivery system an attachment tab of the plurality of attachment tabs is disposed within a circumferentially-extending groove of the plurality of circumferentially-extending grooves and the capsule covers and constrains the valve prosthesis in a radially collapsed configuration, with the capsule extending over the plurality of circumferentially-extending grooves and the attachment tabs received therein.

9. The system of claim 8, wherein the leg of each attachment tab of the plurality of attachment tabs extends generally parallel to the longitudinal axis of the delivery system.

10. The system of claim 8, wherein the leg of each attachment tab of the plurality of attachment tabs extends at an acute angle relative to the longitudinal axis of the delivery system.

11. The system of claim 8, wherein the bend of each attachment tab of the plurality of attachment tabs has a curved profile.

12. The system of claim 8, wherein the bend of each attachment tab of the plurality of attachment tabs forms an angle between the leg and the base, the angle being between 80 and 100 degrees.

13. The system of claim 8, wherein each attachment tab of the plurality of attachment tabs includes a spherical ball disposed at a free end thereof, the spherical ball being attached to the base.

14. The system of claim 8, wherein a number of the attachment tabs is equal to a number of circumferentially-extending grooves.

15. A method of coupling a valve prosthesis to a delivery system for delivery thereof, the method comprising:
positioning a plurality of attachment tabs of the valve prosthesis into an annular groove of a piston of the delivery system, the valve prosthesis including a frame and a prosthetic valve disposed within the frame, the frame being self-expanding and including the plurality of attachment tabs extending from a first end of the frame; and
positioning at least a portion of the valve prosthesis into a capsule of the delivery system such that the capsule covers and constrains the valve prosthesis in a radially collapsed configuration, with the capsule extending over the annular groove of the piston and the attachment tabs received therein.

16. The method of claim 15, wherein each attachment tab of the plurality of attachment tabs has an L-shaped configuration and includes a leg, a base, and a bend extending between the leg and the base, the base extending generally perpendicular to a longitudinal axis of the delivery system, and wherein the base of each attachment tab of the plurality of attachment tabs extends into the annular groove of the piston after the step of positioning the plurality of attachment tabs of the valve prosthesis into the annular groove of the piston of the delivery system.

17. The method of claim 16, wherein the leg of each attachment tab of the plurality of attachment tabs extends generally parallel to the longitudinal axis of the delivery system after the step of positioning the plurality of attachment tabs of the valve prosthesis into the annular groove of the piston of the delivery system.

18. A method of coupling a valve prosthesis to a delivery system for delivery thereof, the method comprising:
positioning a plurality of attachment tabs of the valve prosthesis into a plurality of circumferentially-extending grooves of a piston of the delivery system, the valve prosthesis including a frame and a prosthetic valve disposed within the frame, the frame being self-expanding and including the plurality of attachment tabs extending from a first end of the frame, each attachment tab of the plurality of attachment tabs having an L-shaped configuration and including a leg, a base, and a bend extending between the leg and the base, the base having a first length and extending generally perpendicular to a longitudinal axis of the delivery system, the base having a first length and each circumferentially-extending groove having a length that is at least 300% greater than the first length; and
positioning at least a portion of the valve prosthesis into a capsule of the delivery system such that the capsule covers and constrains the valve prosthesis in a radially collapsed configuration, with the capsule extending over the plurality of circumferentially-extending grooves of the piston and the attachment tabs received therein.

19. The method of claim 18, wherein the base of each attachment tab of the plurality of attachment tabs extends into a circumferentially-extending groove of the plurality of circumferentially-extending grooves of the piston after the step of positioning the plurality of attachment tabs of the valve prosthesis into the plurality of circumferentially-extending grooves of the piston of the delivery system.

20. The method of claim 19, wherein the leg of each attachment tab of the plurality of attachment tabs extends generally parallel to the longitudinal axis of the delivery system after the step of positioning the plurality of attachment tabs of the valve prosthesis into the plurality of circumferentially-extending grooves of the piston of the delivery system.
